SEQUENCE LISTING (44 sequences)

SEQ ID NO: 1 - protein, 183 aa, Homo sapiens
  human OX40 lignad (wt)
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 2 - protein, 8 aa, Artificial Sequence
  linker
GSGSGNGS

SEQ ID NO: 3 - protein, 8 aa, Artificial Sequence
  linker
GSGSGSGS

SEQ ID NO: 4 - protein, 8 aa, Artificial Sequence
  linker
GGSGSGSG

SEQ ID NO: 5 - protein, 6 aa, Artificial Sequence
  linker
GGSGSG

SEQ ID NO: 6 - protein, 4 aa, Artificial Sequence
  linker
GGSG

SEQ ID NO: 7 - protein, 8 aa, Artificial Sequence
  linker
GGSGNGSG

SEQ ID NO: 8 - protein, 8 aa, Artificial Sequence
  linker
GGNGSGSG

SEQ ID NO: 9 - protein, 6 aa, Artificial Sequence
  linker
GGNGSG

SEQ ID NO: 10 - protein, 6 aa, Artificial Sequence
  linker
GSGSGS

SEQ ID NO: 11 - protein, 4 aa, Artificial Sequence
  linker
GSGS

SEQ ID NO: 12 - protein, 3 aa, Artificial Sequence
  linker
GSG

SEQ ID NO: 13 - protein, 218 aa, Artificial Sequence
  human IGG1 Fc N297S
PAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 217 aa, Artificial Sequence
  human IGG1 Fc (wt)
PAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 676 aa, Artificial Sequence
  PROTEIN A (OX40 ligand fused to dglyco Fc)
METDTLLVFVLLVWVPAGNGRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSSSSSSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSSSSSSAWSHPQFEK

SEQ ID NO: 16 - protein, 21 aa, Artificial Sequence
  hinge linker
GSSSSSSSSGSCDKTHTCPPC

SEQ ID NO: 17 - protein, 20 aa, Artificial Sequence
  universal signal peptide
METDTLLVFVLLVWVPAGNG

SEQ ID NO: 18 - protein, 15 aa, Artificial Sequence
  serine linker with strep tag
SSSSSSAWSHPQFEK

SEQ ID NO: 19 - protein, 20 aa, Artificial Sequence
  hinge linker
GSSSSSSSGSCDKTHTCPPC

SEQ ID NO: 20 - protein, 19 aa, Artificial Sequence
  hinge linker
GSSSSSSGSCDKTHTCPPC

SEQ ID NO: 21 - protein, 18 aa, Artificial Sequence
  hinge linker
GSSSSSGSCDKTHTCPPC

SEQ ID NO: 22 - protein, 16 aa, Artificial Sequence
  hinge linker
GSSSGSCDKTHTCPPC

SEQ ID NO: 23 - protein, 18 aa, Artificial Sequence
  hinge linker
GSSSGSCDKTHTCPPCGS

SEQ ID NO: 24 - protein, 20 aa, Artificial Sequence
  hinge linker
GSSSGSCDKTHTCPPCGSGS

SEQ ID NO: 25 - protein, 662 aa, Artificial Sequence
  Prot A (without strep tag)
METDTLLVFVLLVWVPAGNGRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSSSSSSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 661 aa, Artificial Sequence
  receptor binding domains of wt-OX40L fused to Seq14
METDTLLVFVLLVWVPAGNGRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSSSSSSSSGSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 642 aa, Artificial Sequence
  receptor binding domains of wt-OX40L fused to Seq13
RYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSSSSSSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 656 aa, Artificial Sequence
  RBDs wt-OX40L fused to Seq13 (incl. sterp tag)
RYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSSSSSSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSSSSSSAWSHPQFEK

SEQ ID NO: 29 - protein, 641 aa, Artificial Sequence
  RBDs of wt-OX40L fused to Seq14
RYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSSSSSSSSGSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 642 aa, Artificial Sequence
  RBDs of Seq39 fused to deglyco Fc (Seq13)
RSPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRSPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRSPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSSSSSSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 638 aa, Artificial Sequence
  RBDs of Seq39 with one short hinge linker fused to Seq13
RSPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRSPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSRSPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSSSSSSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 639 aa, Artificial Sequence
  RBDs of Seq39 with one shorter hinge linker fused to Seq13
RSPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRSPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRSPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSSSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 637 aa, Artificial Sequence
  RBDs of Seq41 fused to hinge linker and Fc of Seq13
QPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSSSSSSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 635 aa, Artificial Sequence
  RBDs of Seq42 fused to hinge linker and Fc of of Seq13
QPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSSSSSSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 638 aa, Artificial Sequence
  RBDs of Seq44 fused to hinge linker and Fc of Seq13
RYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRYPRIQSIKVQFTEYKKEKGFILTSQKEDEIMKVQNNSVIINSDGFYLISLKGYFSQEVNISLHYQKDEEPLFQLKKVRSVNSLMVASLTYKDKVYLNVTTDNTSLDDFHVNGGELILIHQNPGEGSSSSSSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 403 aa, Artificial Sequence
  exemplary scOX40L-RBD module
RYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 37 - DNA, 2033 bp, Artificial Sequence
  DNA sequence encoding the ORF of Seq25
aagctttagggataacagggtaatagccgccaccatggagactgacaccctgctggtgttcgtgctgctggtctgggtgcctgcaggaaatggaaggtatcccaggattcaaagcatcaaggtgcagttcacagaatataagaaggagaagggatttatcctgaccagccaaaaggaggacgagatcatgaaagtgcaaaataacagcgtcatcattaattgcgacggcttctacctcatctccctgaagggctatttttcccaagaggtgaacatctccctgcactaccaaaaagacgaggagcccctctttcaactgaagaaagtgcggtccgtgaactccctgatggtggcttccctgacctataaggacaaagtgtatctgaatgtgaccaccgataacacctccctggatgatttccatgtgaacggaggcgaactgatcctgatccaccagaaccctggcgaattttgcgtgctgggctccggatctggtaacggttctcggtaccccaggattcagtccattaaggtccaattcaccgagtacaagaaagagaagggcttcatcctcacctcccaaaaggaagatgagattatgaaggtgcagaataatagcgtcattattaattgtgacggattctatctgatctccctgaaaggctatttcagccaggaggtgaatatctccctgcattaccaaaaagatgaggagcctctcttccagctgaaaaaagtgaggtccgtgaattccctgatggtggcctccctgacctacaaagataaggtgtatctgaacgtgaccaccgacaacacaagcctggatgacttccacgtgaatggaggagagctgatcctgattcaccagaatcccggagagttttgcgtcctgggcagcggttctggtaacggctctagatatccccgtattcaaagcatcaaagtccagtttaccgagtacaaaaaggagaaaggattcatcctgaccagccagaaagaagacgagattatgaaagtgcagaacaatagcgtcatcatcaactgcgatggcttttacctgattagcctgaagggctactttagccaggaagtgaatatcagcctgcattatcagaaggacgaagaacctctctttcagctgaaaaaggtgcggagcgtgaacagcctcatggtggccagcctgacctataaagacaaggtgtacctgaatgtcaccaccgataatacctccctggacgactttcatgtgaatggaggcgaactgatcctgatccatcaaaatcccggcgaattttgcgtcctgggatcctcgagttcatcgtcctcatccggctcatgtgataagacccacacctgccctccctgtcctgcccctgagctgctgggcggaccttctgtgttcctgttcccccccaagcctaaggacaccctgatgatctccaggacccctgaggtgacctgtgtggtggtggacgtgtctcacgaagatcccgaggtgaagttcaactggtacgtggacggcgtggaggtccacaacgccaagaccaagcctagggaggagcagtacagctccacctaccgggtggtgtctgtgctgaccgtgctgcaccaggattggctgaacggaaaggagtataagtgtaaggtctccaacaaggccctgcctgcccccatcgagaaaaccatctccaaggccaagggccagcctcgggagcctcaggtgtacaccctgcctcctagcagggaggagatgaccaagaaccaggtgtccctgacctgtctggtgaagggcttctacccttccgatatcgccgtggagtgggagtctaatggccagcccgagaacaactacaagaccacccctcctgtgctggactctgacggctccttcttcctgtactccaagctgaccgtggacaagtccagatggcagcagggcaacgtgttctcctgctccgtgatgcacgaggccctgcacaatcactacacccagaagtccctgtctctgagtccgggcaagtaataggcgcgcc

SEQ ID NO: 38 - protein, 206 aa, Artificial Sequence
  OX40 receptor binding domaine fused to RB69-FOLDON
METDTLLVFVLLVWVPAGNGRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSSGSSGSSGSGYIEDAPSDGKFYVRKDGAWVELPTASGPSSSSSSAWSHPQFEK

SEQ ID NO: 39 - protein, 403 aa, Artificial Sequence
  Exemplary scOX40L-RBD module
RSPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRSPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRSPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 40 - protein, 399 aa, Artificial Sequence
  Exemplary scOX40L-RBD module
RYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 41 - protein, 398 aa, Artificial Sequence
  Exemplary scOX40L-RBD module
QPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 42 - protein, 396 aa, Artificial Sequence
  Exemplary scOX40L-RBD module
QPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 43 - protein, 400 aa, Artificial Sequence
  Exemplary scOX40L-RBD module
GPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSGPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSGPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 44 - protein, 399 aa, Artificial Sequence
  Exemplary scOX40L-RBD module
RYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGSGSGNGSRYPRIQSIKVQFTEYKKEKGFILTSQKEDEIMKVQNNSVIINSDGFYLISLKGYFSQEVNISLHYQKDEEPLFQLKKVRSVNSLMVASLTYKDKVYLNVTTDNTSLDDFHVNGGELILIHQNPGE

(19) United States
(12) Patent Application Publication
(10) Pub. No.: US 2018/0230196 A1
(43) Pub. Date: Aug. 16, 2018

(54) SINGLE-CHAIN OX40-RECEPTOR AGONIST PROTEINS

(71) Applicant: Apogenix AG, Heidelberg (DE)

(72) Inventors: Christian GIEFFERS, Dossenheim (DE); Oliver HILL, Neckarsteinach (DE); Meinolf THIEMANN, Schriesheim (DE); Tim SCHNYDER, Igersheim (DE)

(21) Appl. No.: 15/954,841

(22) Filed: Apr. 17, 2018

Related U.S. Application Data
(63) Continuation of application No. PCT/EP2016/075540, filed on Oct. 24, 2016.
(60) Provisional application No. 62/245,678, filed on Oct. 23, 2015.

(51) Int. Cl. C07K 14/705; C07K 7/06

(52) U.S. Cl. CPC C07K 14/70575; C07K 2319/30; C07K 7/06

(57) ABSTRACT

Provided herein are specific OX40 receptor agonist proteins, nucleic acids encoding the same, and methods of treating a subject having an OX40L-associated disease or disorder. The OX40 receptor agonist proteins provided herein comprise three soluble OX40L domains and an FE fragment. The OX40 receptor agonist proteins are substantially non-aggregating and suitable for therapeutic, diagnostic and/or research applications.

This application is a continuation of PCT/EP2016/075540, filed Oct. 24, 2016; which claims priority to U.S. Provisional Application No. 62/245,678, filed Oct. 23, 2015. The contents of the above applications are incorporated herein by reference in their entirety.

### REFERENCE TO SEQUENCE LISTING, TABLE OR COMPUTER PROGRAM

The Sequence Listing is concurrently submitted herewith with the specification as an ASCII formatted text file via EFS-Web with a file name of Sequence_Listing.txt with a creation date of Apr. 13, 2018, and a size of 101 kilobytes. The Sequence Listing filed via EFS-Web is part of the specification and is hereby incorporated in its entirety by reference herein.

### FIELD OF THE INVENTION

The present invention provides specific OX40 receptor agonist proteins comprising three soluble OX40L domains and an Fc fragment, nucleic acid molecules encoding the OX40 receptor agonist proteins, and uses thereof. The OX40 receptor agonist proteins are substantially non-aggregating and suitable for therapeutic, diagnostic and/or research applications.

### BACKGROUND OF THE INVENTION

It is known that trimerization of TNF superfamily (TNFSF) cytokines is required for efficient receptor binding and activation. Trimeric complexes of TNF superfamily cytokines, however, are difficult to prepare from recombinant monomeric units. WO 01/49866 and WO 02/09055 disclose recombinant fusion proteins comprising a TNF cytokine and a multimerization component, particularly a protein from the C1q protein family or a collectin. A disadvantage of these fusion proteins is, however, that the trimerization domain usually has a large molecular weight and/or that the trimerization is rather inefficient.

Schneider et al. (J Exp Med 187 (1989), 1205-1213) describe that trimers of TNF cytokines are stabilized by N-terminally positioned stabilization motifs. In CD95L, the stabilization of the receptor binding domain trimer is presumably caused by N-terminal amino acid domains which are located near the cytoplasmic membrane.

Shiraishi et al. (Biochem Biophys Res Commun 322 (2004), 197-202) describe that the receptor binding domain of CD95L may be stabilized by N-terminally positioned artificial α-helical coiled-coil (leucine zipper) motifs. It was found, however, that the orientation of the polypeptide chains to each other, e.g. parallel or antiparallel orientation, can hardly be predicted. Further, the optimal number of heptad-repeats in the coiled-coil zipper motif are difficult to determine. In addition, coiled-coil structures have the tendency to form macromolecular aggregates after alteration of pH and/or ionic strength.

WO 01/25277 relates to single-chain oligomeric polypeptides which bind to an extracellular ligand binding domain of a cellular receptor, wherein the polypeptide comprises at least three receptor binding sites of which at least one is capable of binding to a ligand binding domain of the cellular receptor and at least one is incapable of effectively binding to a ligand binding domain of the cellular receptor, whereby the single-chain oligomeric polypeptides are capable of binding to the receptor, but incapable of activating the receptor. For example, the monomers are derived from cytokine ligands of the TNF family, particularly from TNF-α.

WO 2005/103077 discloses single-chain fusion polypeptides comprising at least three monomers of a TNF family ligand member and at least two peptide linkers that link the monomers of the TNF ligand family members to one another. Recent experiments, however, have shown that these single-chain fusion polypeptides show undesired aggregation.

WO 2010/010051 discloses single-chain fusion polypeptides comprising three soluble TNF family cytokine domains and at least two peptide linkers. The described fusion polypeptides are substantially non-aggregating.

Recent studies have shown that the in vivo anti tumor activity of an anti-OX40-mAb is dependent on Fc-gamma-R driven mechanisms and does not rely on agonistic activity only.

Bulliard, Y., R. Jolicoeur, J. Zhang, G. Dranoff, N. S. Wilson and J. L. Brogdon (2014). “OX40 engagement depletes intratumoral Tregs via activating FcgammaRs, leading to antitumor efficacy,” Immunol Cell Biol 92(6): 475-480.

There is a need in the art for novel OX40 receptor agonists that exhibit high biological activity independent of Fc-gamma-R based crosslinking in vivo, high stability, and allow for efficient recombinant manufacturing.

### SUMMARY OF THE INVENTION

The present invention provides specific OX40 receptor agonist proteins that mimic the OX40:OX40L interaction in vivo, exhibit low proteolytic degradation and a shorter in vivo half life as compared to agonistic monoclonal antibodies.

The OX40 receptor agonist proteins of the instant invention generally comprise:(i) a first soluble OX40L cytokine domain; (ii) a first peptide linker; (iii) a second soluble OX40L domain; (iv) a second peptide linker; (v) a third soluble OX40L domain; (vi) a third peptide linker (e.g., a hinge-linker) and (vii) an antibody Fc fragment.

In one embodiment, the antibody Fc fragment (vii) is located N terminal to the first OX40L domain (i) and/or C-terminal to the third OX40L domain (v). In another embodiment the antibody Fc fragment is located C-terminally to the third OX40L domain (v). In one embodiment, the polypeptide is substantially non-aggregating. In another embodiment, the second and/or third soluble OX40L domain is an N-terminally shortened domain which optionally comprises amino acid sequence mutations.. In another embodiment, the soluble OX40L domains (i), (ii) and (iii) are an C-terminally shortened domain which optionally comprises amino acid sequence mutations. In one embodiment, at least one of the soluble OX40L domains, particularly at least one of the soluble OX40L domains (iii) and (v), is a soluble OX40L domain with an N-terminal sequence which starts at amino acid Gln51 or R55 or R58 of human OX40L and wherein Tyr56 may be replaced by a neutral amino acid, e.g., Ser or Gly. In another embodiment, at least one of the soluble OX40L domains, particularly at least one of the soluble OX40L domains (iii) and (v), is a soluble OX40L domain with an N-terminal sequences selected from (a) Pro57-Arg58 and (b) (Gly/Ser)56-Arg58. In one embodiment, the soluble OX40L domain ends with amino acid Leu183 of human OX40L and/or optionally comprises one or more mutation at positions Y69, L160, 080, N90, C97, N114, E123, T144, Y145, K146, N152, N157, D162, H164, N166, G168, G178, F180 or C181. In one embodiment, the soluble OX40L domains (i), (iii) and (v) comprise amino acids Arg58 Leu183 of human OX40L according to SEQ ID NO: 1.

In one embodiment, at least one of the soluble OX40L domains, particularly at least the soluble OX40L domains (i), is a soluble OX40L domain with an N-terminal sequence which starts at amino acid Tyr56 and wherein Tyr56 may be replaced by Gln, Ser or Gly. In one embodiment, at least one of the soluble OX40L domains, particularly at least the soluble OX40L domain (iii), is a soluble C-terminal shortened OX40L domain ending with Pro177 and comprises a mutation at position C97. In another embodiment, at least one of the soluble OX40L domains, particularly at least the soluble OX40L domains (iii), is a soluble C-terminal shortened OX40L domain ending with Gly178 and comprises a mutation at position C97.In still another embodiment, at least one of the soluble OX40L domains, particularly at least the soluble OX40L domains (iii), is a soluble C-terminal shortened OX40L domain ending with Glu179 and comprises a mutation at position C97. In another embodiment, at least one of the soluble OX40L domains, particularly at least the soluble OX40L domains (iii), is a soluble C-terminal shortened OX40L domain ending with Val182 and comprises a mutation at position C97 and C181.

In one embodiment, the first and second peptide linkers (ii) and (iv) independently have a length of 3-8 amino acids, particularly a length of 3, 4, 5, 6, 7, or 8 amino acids, and preferably are glycine/serine linkers, optionally comprising an asparagine residue which may be glycosylated. In one embodiment, the first and the second peptide linkers (ii) and (iv) consist of the amino acid sequence according to SEQ ID NO: 2. In another embodiment, the polypeptide additionally comprises an N-terminal signal peptide domain, e.g., of SEQ ID NO: 17, which may comprise a protease cleavage site, and/or which additionally comprises a C-terminal element which may comprise and/or connect to a recognition/purification domain, e.g., a Strep-tag attached to a serine linker according to SEQ ID NO: 18.

In one embodiment, the antibody Fc fragment (vii) is fused to the soluble OX40L domain (i) and/or (v) via a hinge-linker, preferably of SEQ ID NO: 16. In another embodiment, the antibody Fc fragment (vii) consists of the amino acid sequence as shown in SEQ ID NO: 13 or 14.

In one embodiment, the single-chain fusion polypeptide of the present invention comprises the amino acid sequence selected from the group consisting of SEQ ID NO: 15, and 25-35.

In one embodiment, the present invention provides an OX40 receptor agonist protein comprising a dimer of two single-chain fusion polypeptides each having the amino acid sequence set forth in SEQ ID NO: 27. In one embodiment, the two polypeptides are covalently linked through three interchain disulfide bonds formed between cysteine residues 415, 421, and 424 of each polypeptide.

In one embodiment, one or more of the asparagine residues at positions 135 and 272 of the mature polypeptide(s) SEQ ID NO: 27, 28, 29, 30, or 35 are N-glycosylated. In another embodiment, the asparagine residues at positions 135 and 272 of the polypeptide(s) are both N-glycosylated. Similar asparagine residues are positions 134 and 269 of SEQ ID NO: 33 and positions 134 and 268 of SEQ ID NO: 34. In another embodiment, only the asparagine residue at position 135 of the mature polypeptides SEQ ID NO: 31 is glycosylated as the asparagine 272 is not present in this protein.

In another embodiment, the polypeptide(s) are further post-translationally modified. In another embodiment, the post-translational modification comprises the N-terminal glutamine of the Y56Q mutein of the first soluble domain (i) modified to pyroglutamate.

### DESCRIPTION OF THE FIGURES

FIG. 1 Domain structure of a single-chain fusion polypeptide comprising three OX40L domains. I., II., III. Soluble OX40L domains.

FIG. 2 Schematic picture representing the general structure of OX40L.
▪▪▪ Cell membrane, N-terminus located within the cell,
1, anti-parallel β-fold of receptor-binding domain (RBD),
2. interface of RBD and cell membrane,
3. protease cleavage site.

FIG. 3 Single-chain fusion polypeptide comprising an additional Fab antibody fragment.

FIG. 4 Dimerization of two C-terminally fused single-chain Fc fusion polypeptides via three disulfide bridges.

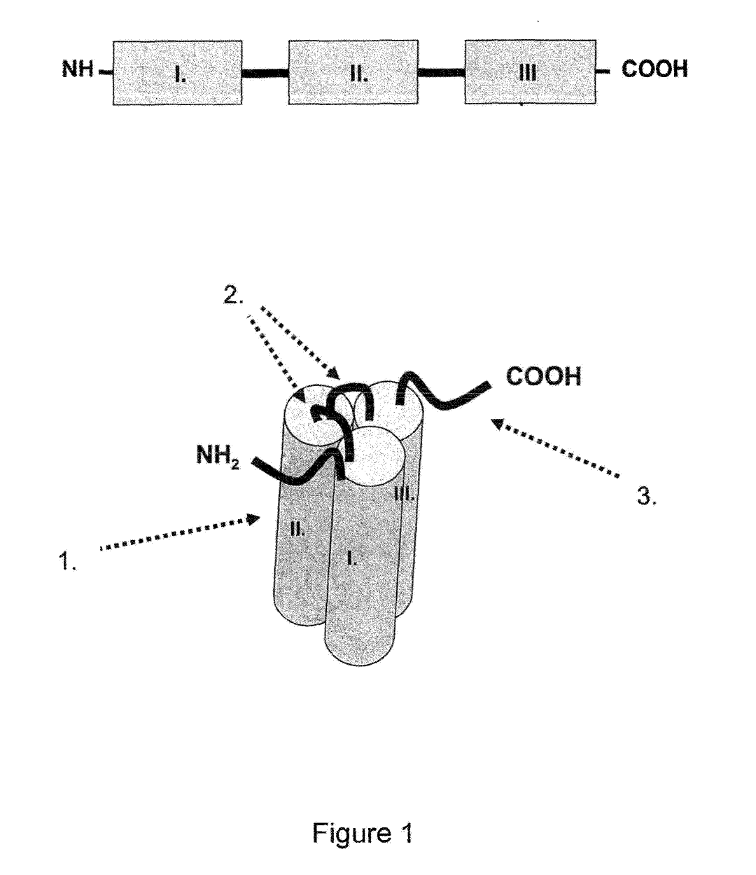

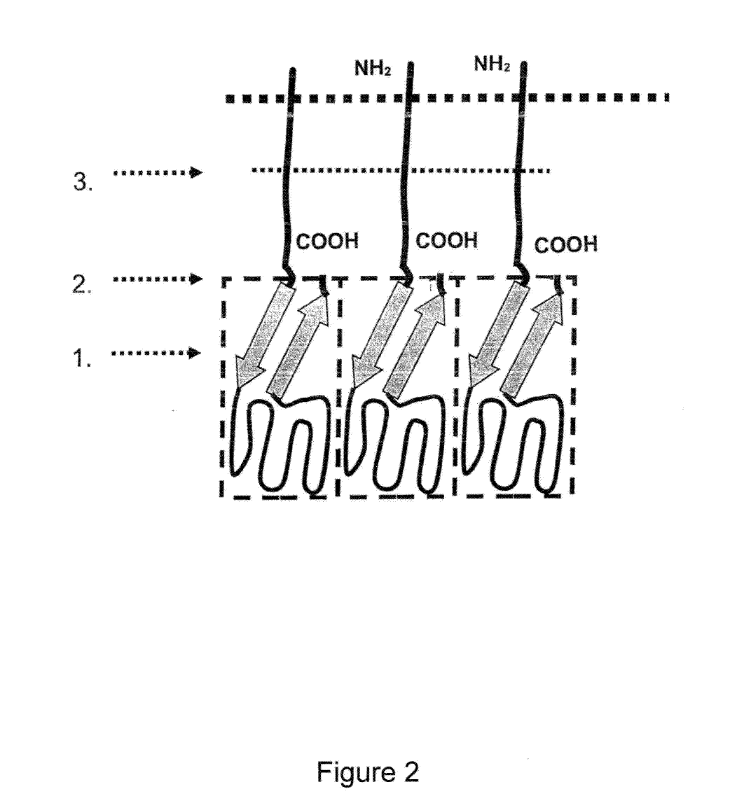

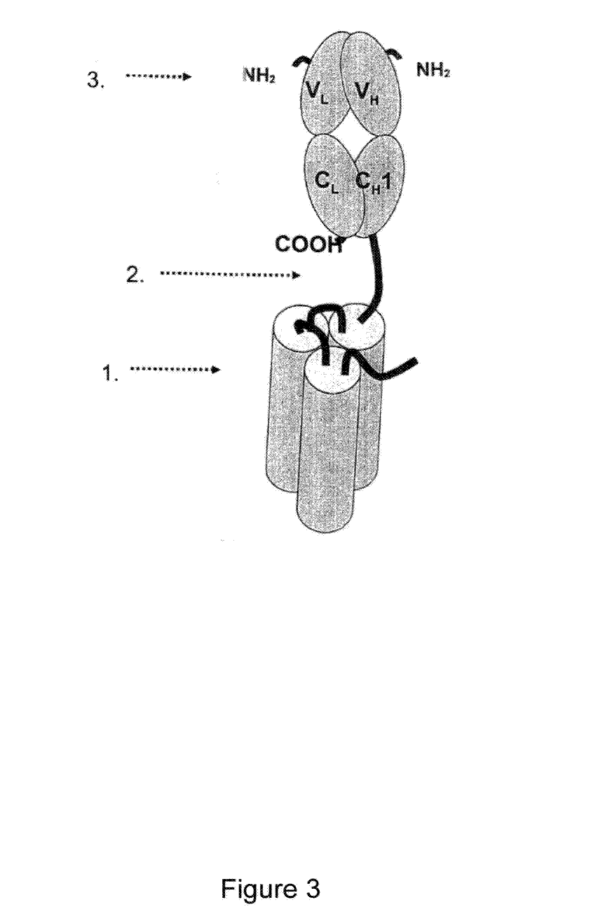

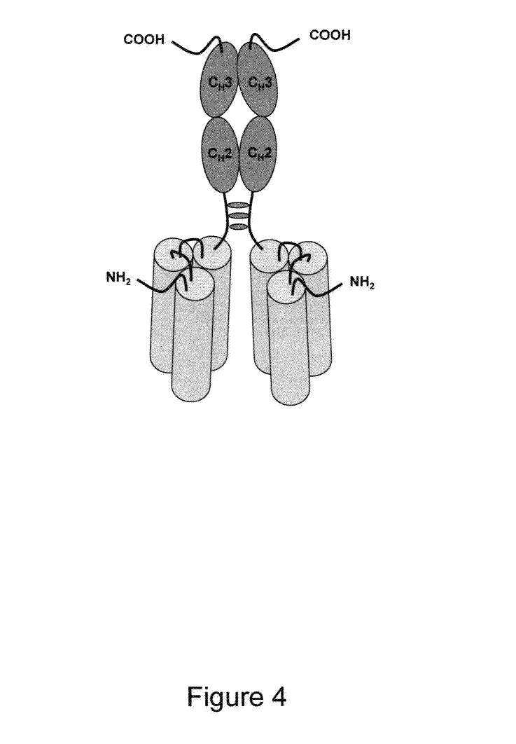

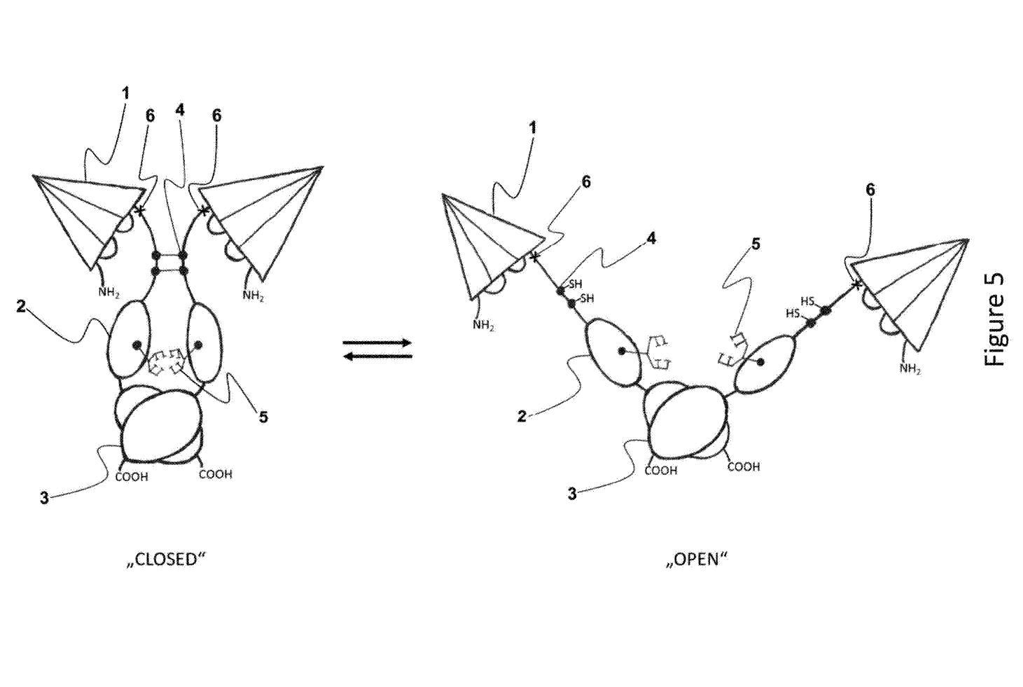

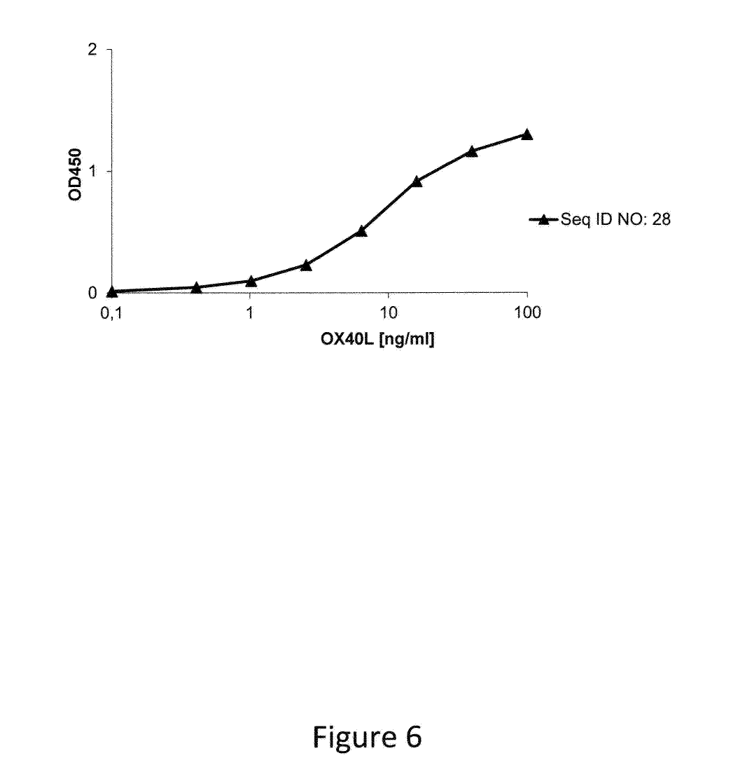

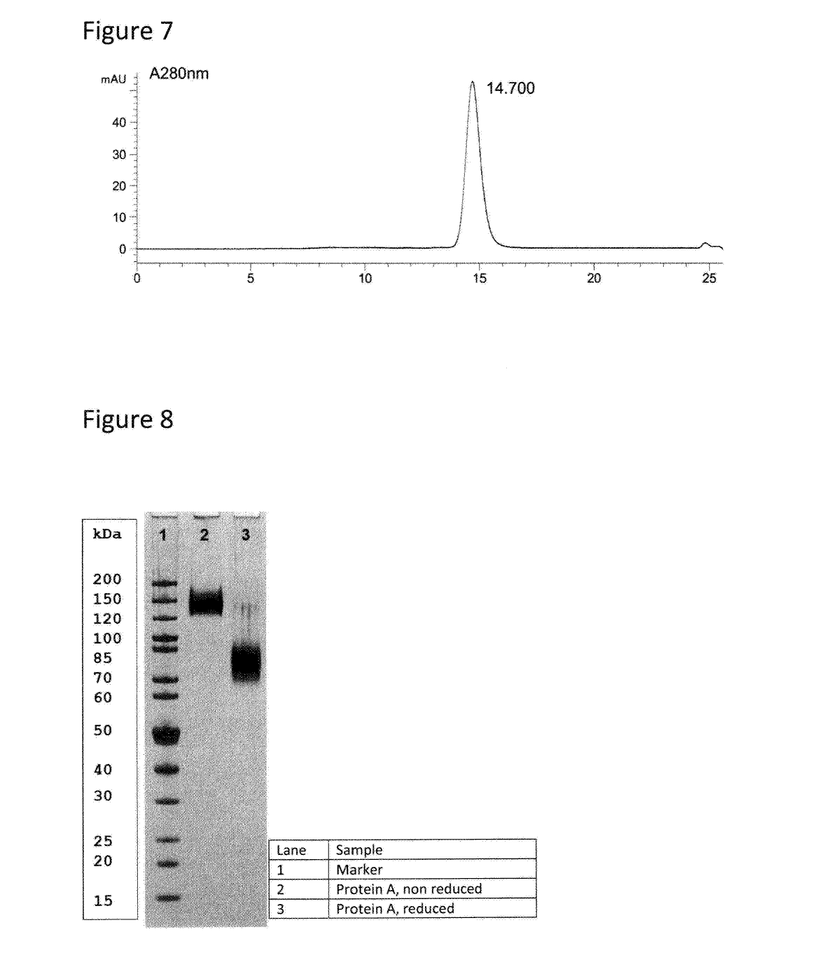

FIG. 5 Schematic representation of the hexavalent single chain CD27 receptor agonist fusion protein of the invention. CH2-Carbohydrates (5) present on the inner surface areas normally shield the CH2-subdomain sterically (2) from proteases during “open Fc-conformation transits” wherein hinge-interchain disulfide bonds (4) are reduced and the covalent interchain linkage is disrupted. This enables CH2-dissociation and exposure of the inner surface areas and the upper hinge lysine K223 (6) towards proteases. Dimer association in the “open stage” remains intact due to the high affinity of the CH3 domains (3) to each other.
(1) scCD27L-RBD; (2) CH2 domain; (3) CH3 domain; (4) Hinge-Cysteines (left side: oxidized to disulfidbridges; right side reduced stage with free thiols); (5) CH2-Carbohydrates attached to N297 position (EU-numbering); (6) Upper Hinge Lysine (K223)

FIG. 6 ELISA assessing the binding of OX40 receptor agonist protein (Protein A) to its receptor

FIG. 7 Analytical size exclusion chromatography of strep tagged PROTEIN A (SEQ ID NO: 28) performed on a 1260 Infinity HPLC system using a Tosoh TSKgelG3000SWxlcolumn. The column was loaded with protein at a concentration of 0.8 mg/ml in a total volume of 20 μl. The flow rate was set to 0.5 ml/min. One observes a single main peak at 14.7 min for PROTEIN A

FIG. 8 SDS-PAGE results of PROTEIN A under non-reducing and reducing conditions. 240 ng of PROTEIN A were loaded on an SDS-PAGE 4-12% Bis-Tris gel under non-reducing (lane 2) or reducing (lane 3) conditions containing DTT as reducing agent. Gels were run at 110V for 20 min followed by 190V for 60 min and were subsequently stained using a silver-stain protocol. One observes a molecular weight difference between the main bands in lane 2 and lane 3 of about 70-80 kDa. As this is about half the molecular weight as observed for the main band in lane 2, this indicates that the homodimer in lane 2 is covalently linked by disulfide bridges. The bonds are lost under reducing conditions in lane 3.

### DETAILED DESCRIPTION OF THE INVENTION

The present invention provides a single-chain fusion polypeptide comprising at least three soluble OX40L domains connected by two peptide linkers and N-terminally and/or C-terminally an antibody-derived dimerization domain. The inventors have discovered that dimerization of the two single-chain fusion polypeptides through the dimerization domain results in a hexavalent OX40 receptor agonist, which provides high biological activity and good stability.

Preferably, the single-chain fusion polypeptide is non-aggregating. The term “non-aggregating” refers to a monomer content of the preparation of 50%, preferably 70% and more preferably 90%, The ratio of monomer content to aggregate content may be determined by examining the amount of aggregate formation using size-exclusion chromatography (SEC). The stability concerning aggregation may be determined by SEC after defined time periods, e.g. from a few to several days, to weeks and months under different storage conditions, e.g. at 4° C. or 25° C. For the fusion protein, in order to be classified as substantially non-aggregating, it is preferred that the “monomer” content is as defined above after a time period of several days, e.g. 10 days, more preferably after several weeks, e.g., 2, 3 or 4 weeks, and most preferably after several months, e,g. 2 or 3 months of storage at 4° C., or 25° C. With regard to the definition of “monomer” in the case of FC-fusion proteins, the assembly of two polypeptide chains is driven by the FC-part and the functional unit of the resulting assembled protein consists of two chains. This unit is defined as “monomer” in the case of Fc-fusion proteins regardless of being a dimerized single-chain fusion polypeptide.

The single-chain fusion polypeptide may comprise additional domains which may be located at the N- and/or C-termini thereof. Examples for additional fusion domains are e.g. an N-terminal signal peptide domain which may comprise a protease cleave site or a C-terminal element which may comprise and/or connect to a recognition/purification domain. According to a preferred embodiment, the fusion polypeptide comprises a Strep-tag at its C-terminus that is fused via a linker. An exemplary Strep-tag including a short serine linker is shown in SEQ ID NO: 18.

The OX40 receptor agonist protein of the present invention comprises three soluble domains derived from OX40L. Preferably, those soluble domains are derived from a mammalian, particularly human OX40L including allelic variants and/or derivatives thereof. The soluble domains comprise the extracellular portion of OX40L including the receptor binding domain without membrane located domains. Like other proteins of the TNF superfamily, OX40L is anchored to the membrane via an N-terminal portion of 15-30 amino acids, the so-called stalk-region. The stalk region contributes to trimerization and provides a certain distance to the cell membrane. However, the stalk region is not part of the trimeric receptor binding domain (RBD) with the receptor binding sites located at the protomer interfaces.

Importantly, the RBD is characterized by a particular localization of its N- and C-terminal amino acids. Said amino acids are immediately adjacent and are located in close proximity to the axis of the trimer. The first N-terminal amino acids of the RBD form an anti-parallel beta-strand with a C-terminal region of the RBD ending in the case of human Ox40L with His174, Human Ox40L contains a C-terminal extension (Q175-L183) fixed via a disulfidbridge between Cys97 and Cys181 to the tip of the protomer. The C-terminal Leu183 is in close proximity to Arg58 of each protomer.

Thus, the aforementioned anti-parallel beta-strand of the RBD and the C-terminal extension form an interface with the cell membrane, which is connected to and anchored within the cell membrane via the amino acids of the stalk region. It is highly preferred that the soluble OX40L domains of the OX40 receptor agonist protein comprise a receptor binding domain of the OX40L lacking any amino acids from the stalk region. Otherwise, a long linker connecting the C-terminus of one of the soluble domains with the N-terminus of the next soluble domain would be required to compensate for the N-terminal stalk-region of the next soluble domain, which might result in instability and/or formation of aggregates.

A further advantage of such soluble domains is that the N-terminal amino acids of the RBD are not accessible for any anti-drug antibodies. Preferably, the single-chain fusion polypeptide consisting of (i) a first soluble OX40L cytokine domain; (ii) a first peptide linker; (iii) a second soluble OX40L domain; (iv) a second peptide linker; (v) a third soluble OX40L domain is capable of forming an ordered structure mimicking the trimeric organization of its natural counterpart thereby comprising at least one functional binding site for the respective OX40L receptor. The single-chain fusion polypeptide comprising components (i)-(v) is therefore also termed single-chain-OX40L-receptor-binding-domain (scOX40L-RBD),

The OX40 receptor agonist protein comprises three functional OX40 receptor binding sites, i.e. amino acid sequences capable of forming a complex with a OX40 receptor. Thus, the soluble domains are capable of binding to the corresponding OX40 receptor. In one embodiment, at least one of the soluble domains is capable of receptor activation, whereby apoptotic and/or proliferative activity may be affected. In a further embodiment, one or more of the soluble domains are selected as not being capable of receptor activation.

The soluble OX40L domain may be derived from human OX40L as shown in SEQ ID NO: 1. Preferably, the soluble OX40L domains are derived from human OX40L, particularly starting from amino acids 55, 56, 57 or 58 and comprise particularly amino acids 55-183 or 56-183 or 57-183 or 58-183 of SEQ ID NO: 1. Optionally, amino acid Tyr56 of SEQ ID NO: 1 may be replaced by a non-charged amino acid, e.g. Ser or Gly or is replaced by Glutamine.

TABLE 1
Sequence of Wild-Type Human OX40L Protein

| SEQ | |
|---|---|
| ID | |
| NO | Sequence |
| 1 | MERVQPLEENVGNAARPRFERNKLLLVASVIQGLGLLLCFTYICL |
| | HFSALQVSHRYPRIQSIKVQFTEYKKEKGFILTSQKEDEIMKVQN |
| | NSVIINCDGFYLISLKGYFSQEVNISLHYQKDEEPLFQLKKVRSV |
| | NSLMVASLTYKDKVYLNVTTDNTSLDDFHVNGGELILIHQNPGEF |
| | CVL |

As indicated above, the soluble OX40L domains may comprise the wild-type sequences as set forth in SEQ ID NO: 1. It should be noted, however, that it is possible to introduce mutations in one or more of these soluble domains, e.g. mutations which alter (e.g. increase or decrease) the binding properties of the soluble domains. In one embodiment, soluble domains that cannot bind to the corresponding cytokine receptor can be selected.

In a further embodiment of the invention, the soluble OX40L domain (i) comprises a mutant of OX40L or a receptor binding domain thereof resulting in reduced affinity and/or reduced activation of OX40 receptor.

OX40L-Muteins Affecting Receptor Binding and/or Activity

The mutant may be generated by any technique known by a skilled person. The substitution may affect at least one amino acid of OX40L, e.g., human OX40L (e.g., SEQ ID NO: 1) or a receptor binding domain thereof as described herein. Preferred substitutions in this regard affect at least one of the following amino acids of human OX40L of SEQ ID NO: 1: Y69, Q80, N90, C97, N114, E123, T144, Y145, K146, N152, N157, L160, D162, H164, N166, G168, G178, F180 and C181. In a preferred embodiment H164 is mutated to R, D, E, Q or N and/or Y145 is mutated to S, D, E or R. In another preferred embodiment, the C-terminal region F180-L181 is deleted and simultaneously C97 mutated to serine (C97S) from at least one of the soluble domains (i), (III) or (v).

The amino acid substitution(s) may affect the binding and/or activity of OX40L, e.g., human OX40L, to or on either the OX40 binding or the OX40 induced signaling. The binding and/or activity of the OX40 may be affected positively, i.e., stronger, more selective or more specific binding and/or more activation of the receptor. Alternatively, the binding and/or activity of the OX40 may be affected negatively, i.e., weaker, less selective or less specific binding and/or less or no activation of the receptor.

Thus one embodiment is an OX40 receptor agonist protein as described herein wherein at least one of the soluble domains comprises a mutant of OX40L or a receptor binding domain thereof which binds and/or activates OX40 to a lesser extent than the wildtype-OX40L,

OX40L-Muteins with Enhanced Stability/Solubility

In a further embodiment of the invention, one or more of the soluble OX40L domains (i), (iii), and (v) may comprise a mutant of OX40L or a receptor binding domain thereof resulting in reduced self-aggregation and/or prolonged in vivo stability.

Preferred substitutions in this regard are N90[S, C], N114[S or D] and N156[S or D]. The mutation(s) of each OX40L domain may be the same or different.

The single-chain fusion molecule of the present invention comprises three soluble OX40L domains, namely components (i), (iii) and (v). The stability of a single-chain OX40L fusion polypeptide against aggregation is enhanced, if the second and/or third soluble OX40L domain is an N-terminally shortened domain which optionally comprises amino acid sequence mutations. Thus, preferably, both the second and the third soluble OX40L domain are N-terminally shortened domains which optionally comprise amino acid sequence mutations in the N-terminal regions, preferably within the first five amino acids of the N-terminus of the soluble OX40L domain. These mutations may comprise replacement of basic amino acids, by neutral amino acids, particularly serine or glycine,

In contrast thereto, the selection of the first soluble OX40L domain is not as critical. Here, a soluble domain having a full-length N-terminal sequence may be used. It should be noted, however, that also the first soluble OX40L domain may have an N-terminally shortened and optionally mutated sequence.

In a further preferred embodiment of the present invention, the soluble OX40L domains (i), (iii) and (v) are soluble human OX40L domains. The first soluble OX40L domain (i) may be selected from native, shortened and/or mutated sequences. Thus, the first soluble OX40L domain (i) has an N-terminal sequence which may start at amino acid Arg55 or Tyr56 of human OX40L, and wherein Tyr56 may be replaced by a neutral amino acid, e.g. by Ser or Gly or by Gln to enable pyroglutamate formation during expression. The second and third soluble OX40L domains (iii) and (v) have a shortened N-terminal sequence which preferably starts with amino acid Pro57 or Arg58 of human OX40L (SEQ D NO:1) and wherein Pro57 may be replaced by another amino acid, e.g. Ser or Gly.

Preferably, the N-terminal sequence of the soluble OX40L domains (iii) and (v) is selected from:

(a) Pro57 or Arg58

(b) (Gly/Ser) 57

The soluble OX40L domain preferably ends with amino acid L183 of human OX40L. In certain embodiments, the OX40L domain may comprise internal mutations as described above.

Components (ii) and (iv) of the OX40 receptor agonist protein are peptide linker elements located between components (i) and (iii) or (iii) and (v), respectively. The flexible linker elements have a length of 3-8 amino acids, particularly a length of 3, 4, 5, 6, 7, or 8 amino acids. The linker elements are preferably glycine/serine linkers, i.e. peptide linkers substantially consisting of the amino acids glycine and serine. In cases in in which the soluble cytokine domain starts with S or G (N-terminus), the linker ends before this S or G.

It should be noted that linker (ii) and linker (iv) do not need to be of the same length. In order to decrease potential immunogenicity, it may be preferred to use shorter linkers. In addition it turned out that shorter linkers lead to single chain molecules with reduced tendency to form aggregates. Whereas linkers that are substantially longer than the ones disclosed here may exhibit unfavorable aggregations properties.

If desired, the linker may comprise an asparagine residue which may form a glycosylate site Asn-Xaa-Ser. In certain embodiments, one of the linkers, e.g. linker (ii) or linker (iv) comprises a glycosylation site. In other embodiments, both linkers (iv) comprise glycosylation sites. In order to increase the solubility of the OX40L agonist proteins and/or in order to reduce the potential immunogenicity, it may be preferred that linker (ii) or linker (iv) or both comprise a glycosylation site.

Preferred linker sequences are shown in Table 2. A preferred linker is GSGSGNGS (SEQ ID NO: 2).

TABLE 2
Example Linker Sequences

| SEQ | |
|---|---|
| ID | |
| NO | Sequence |
| 2 | GSGSGNGS |
| 3 | GSGSGSGS |
| 4 | GGSGSGSG |
| 5 | GGSGSG |
| 6 | GGSG |
| 7 | GGSGNGSG |
| 8 | GGNGSGSG |
| 9 | GGNGSG |
| 10 | GSGSGS |
| 11 | GSGS |
| 12 | GSG |

The OX40 receptor agonist protein additionally comprises an antibody Fc fragment domain which may be located N-terminal to the first OX40L domain (i) and/or C-terminal to the third OX40L domain (v). Preferably, the antibody Fc fragment domain comprises a reduced capability to interact with Fc-gamma-R receptors in vivo. Preferably, the antibody Fc fragment domain comprises or consists of an amino acid sequence as shown in SEQ ID NO: 13 or 14 (see Table 3). Sequence ID NO: 13 has N2975 mutation compared to wildtype human IGG1-Fc. Sequence ID NO: 14 is a glycosylated (N297 wildtype) human IGG1 Fc mutein with reduced Fc-gamma-R binding capability.

TABLE 3
Examples of Fc Fragment Domains

| SEQ | |
|---|---|
| ID | |
| NO | Sequence |
| 13 | PAPELLGGPSVFLFPPKPKDTLMISRTPEVTCVVVDVSHEDPEVKF |
| | NWYVDGVEVHNAKTKPREEQYSSTYRVVSVLTVLHQDWLNGKEYKC |
| | KVSNKALPAPIEKTISKAKGQPREPQVYTLPPSREEMTKNQVSLTC |
| | LVKGFYPSDIAVEWESNGQPENNYKTTPPVLDSDGSFFLYSKLTVD |
| | KSRWQQGNVFSCSVMHEALHNHYTQKSLSLSPGK |
| 14 | PAPPVAGPSVFLFPPKPKDTLMISRTPEVTCVVVDVSHEDPEVKFN |
| | WYVDGVEVHNAKTKPREEQYNSTYRVVSVLTVLHQDWLNGKEYKCK |
| | TVSNKGLPSSIEKISKAKGQPREPQVYTLPPSREEMTKNQVSLTCL |
| | VKGFYPSDIAVEWESNGQPENNYKTTPPVLDSDGSFFLYSKLTVDK |
| | SRWQQGNVFSCSVMHEALHNHYTQKSLSLSPGK |

Number of Glycosylation Sites and In Vivo Stability

The total number of glycosylation sites and the individual position of the carbohydrates in three dimensions impacts the in-vivo stability of OX40 receptor agonist proteins. Further, carbohydrate recognition depends on local density of the terminal saccharides, the branching of the carbohydrate tree and the relative position of the carbohydrates to each other matter.

Further, partially degraded carbohydrates reduce the in vivo half-life of OX40 receptor agonist proteins through lectin-driven mechanisms. By reducing the total number of glycosylation sites on the molecule, the resulting compound is less accessible to these mechanisms, increasing half-life.

Depletion of the CH2-domain carbohydrates of the Fc-domain is necessary in order to avoid Fc-receptor based crosslinking in vivo and potential OX40L-receptor superclustering-based toxicity. Also, unwanted Fc-driven mechanisms like ADCC could lead to toxic events. Accordingly, in one embodiment, the overall number of glycosylation sites on the OX40 receptor agonist proteins of the instant invention is reduced through the depletion of CH2 glycosylation sites, particularly the N-glycosylation site, resulting in OX40 receptor agonist proteins comprising N297S equivalent mutations of SEQ ID NO: 15 (PROTEIN A) (according to the EU numbering system) creating aglycosl-CH2 domains. In another embodiment of the invention, one or more of the soluble OX40L domains (i), (iii), and (v) may comprise a N91 and/or N114 exchanged to aspartate, serine or glycine resulting in OX40 receptor agonistic fusion proteins with a reduced number of glycosylation sites. In a preferred embodiment, the N91[D, S, G] and N114[D, S, G] mutations are restricted to the soluble OX40L domains (iii) and (v) of the agonistic OX40 receptor agonistic fusion proteins of the present invention.

CH2-Domain Destabilization is Compensated by an Additional Hinge-Cysteine

CH2-glycosylation present on the inner surface areas normally shields the subdomain from proteases during “open Fc-conformation transits” wherein hinge-interchain disulfide bonds are reduced and the covalent interchain linkage is disrupted (FIG. 5). This enables CH2-dissociation and exposure of the inner surface area towards proteases. OX40 receptor agonist proteins comprising an Fc-domain with a N2975 equivalent mutation of SEQ ID NO: 15 (PROTEIN A) (according to the EU numbering system) creates an aglycosylated-CH2 and are therefore likely to be subject to protease digestion and less stable than equivalent structures with wild-type CH2 glycosylation. This would impact the compound's stability during USP/DSP/storage, where host cell proteases are present and have long-term access to the structure. Accordingly, in certain embodiments, the OX40 receptor agonist lacks CH2 glycosylation sites, but comprises glycosylation sites in the linker sequences of each polypeptide chain (e.g., GSGSGNGS, SEQ ID NO: 2).

According to a preferred embodiment of the invention, the antibody Fc fragment domain is fused via a hinge-linker element. The hinge-linker element has a length of 10-30 amino acids, particularly a length of 15-25 amino acids, e.g. 22 amino acids. The term “hinge-linker” includes any linker long enough to allow the domains attached by the hinge-linker element to attain a biologically active confirmation. The hinge-linker element preferably comprises the hinge-region sequence of an immunoglobulin, herein referred to as “Ig hinge-region”. The term “Ig hinge-region” means any polypeptide comprising an amino acid sequence that shares sequence identity or similarity with a portion of a naturally occurring Ig hinge-region sequence which includes one or more s cysteine residues, e.g., two cysteine residues, at which the disulfide bonds link the two heavy chains of the immunoglobulin.

Derivatives and analogues of the hinge-region can be obtained by mutations. A derivative or analogue as referred to herein is a polypeptide comprising an amino acid sequence that shares sequence identity or similarity with the full length sequence of the wild type (or naturally occurring protein) except that it has one or more amino acid sequence differences attributable to a deletion, insertion and/or substitution.

The number of molecules with open Fc-conformation in an individual OX40 receptor agonist protein depends on the number of interchain-disulfide bonds present in the hinge region. Accordingly, in one embodiment a third cysteine (C225 according to the EU numbering system) was introduced into the hinge region of the OX40 receptor agonist proteins of the instant invention in order to ameliorate the effect of depleting the CH2-glycosites.

### Exchange of a Lysine to Glycine in the Hinge Region Results in Enhanced Proteolytic Stability

In one embodiment, the OX40 receptor agonist proteins of the invention additionally comprise a mutation of the upper-hinge lysine (K223, according to the EU numbering system) to a glycine to reduce proteolytic processing at this site, thereby enhancing the overall stability of the fusion protein. Combining aforementioned introduction of a third cysteine (C225, according to the EU numbering system) with the aforementioned lysine to glycine mutation (K223G, according to the EU numbering system) within the hinge region results in an overall stabilized OX40 receptor agonist protein of the instant invention.

A particularly preferred hinge-linker element including the aforementioned cysteine (C225) and the lysine to glycine mutation (K223G) comprises or consists of the amino acid sequence as shown in SEQ ID NO: 16 (Table 4).

Endogenous Cysteines Interfere with Hinge-Disulfide Formation

The interchain-disulfide connectivity of the hinge region stabilizing the homodimer of the hexavalent OX40 receptor agonist protein is also affected by the free thiol groups of the OX40L subsequences. Free thiol groups can be created through reduction of surface exposed disulfide-bridges, e.g. by reduction of the C97-C181 disulfide of OX40L. This also leads to the aforementioned open FC-conformation due to self-reduction of the hinge disulfide-bridges of the structure by the endogenous free thiols of the preparation at high protein concentrations. In consequence, single-chain OX40L-FC fusion proteins comprising free thiols are expected to be less stable during manufacture and storage, when longtime exposure to oxygen and proteases occurs.

Therefore, to enable manufacture of a hexavalent OX40 receptor agonist at technical scale, the C97 and C181 residues are preferably mutated simultaneously to a different amino-acid (e.g. L, S, A or G).

The OX40 receptor agonist protein may additionally comprise an N-terminal signal peptide domain, which allows processing, e.g. extracellular secretion, in a suitable host cell. Preferably, the N-terminal signal peptide domain comprises a protease cleavage site, e.g. a signal peptidase cleavage site and thus may be removed after or during expression to obtain the mature protein. A particularly preferred N-terminal signal peptide domain comprises the amino acid sequence as shown in SEQ ID NO: 17 (Table 4).

Further, the OX40 receptor agonist protein may additionally comprise a C-terminal element, having a length of e.g. 1-50, preferably 10-30 amino acids which may include or connect to a recognition/purification domain, e.g. a FLAG domain, a Strep-tag or Strep-tag II domain and/or a poly-His domain. According to a preferred embodiment, the fusion polypeptide comprises a Strep-tag fused to the C-terminus via a short serine linker as shown in SEQ ID NO: 18 (Table 4).

Preferred hinge-linker elements (SEQ ID NO: 16, 19-24), a preferred N-terminal signal peptide domain (SEQ ID NO: 17) and serine linker-strep tag (SEQ ID NO: 18) are shown in Table 4.

TABLE 4
Exemplary domains and linkers

| SEQ | |
|---|---|
| ID | |
| NO | Sequence |
| 16 | GSSSSSSSSGSCDKTHTCPPC |
| 17 | METDTLLVFVLLVWVPAGNG |
| 18 | SSSSSSAWSHPQFEK |
| 19 | GSSSSSSSGSCDKTHTCPPC |
| 20 | GSSSSSSGSCDKTHTCPPC |
| 21 | GSSSSSGSCDKTHTCPPC |
| 22 | GSSSGSCDKTHTCPPC |
| 23 | GSSSGSCDKTHTCPPCGS |
| 24 | GSSSGSCDKTHTCPPCGSGS |

In one embodiment of the invention, the fusion polypeptide comprises three soluble OX40L domains fused by peptide linker elements of SEQ ID NO: 2. All three soluble OX40L domain (i), (iii), (v) consists of amino acids 55-183 of human OX40L according to SEQ ID NO: 1. The resulting scOX40L-RBD sequence module is shown in table 5b SEQ ID NO: 36.

In a further preferred embodiment of the invention, the fusion polypeptide comprises three soluble OX40L domains fused by peptide linker elements of SEQ ID NO: 2. All three soluble OX40L domain (i), (iii), (v) consists of amino acids 55-183 of human OX40L according to SEQ ID NO: 1 with Y56S mutation. The resulting scOX40L-RBD sequence module is shown in table 5b SEQ ID NO: 39.

In another embodiment of the invention, the fusion polypeptide comprises three soluble OX40L domains fused by peptide linker elements of SEQ ID NO: 2. The first soluble OX40L domain (i) consists of amino acids 55-183 of human OX40L according to SEQ ID NO: 1 and the soluble OX40L domains (iii) and (v) consist of amino acids 57-183 of human OX40L according to SEQ ID NO: 1 The resulting scOX40L-RBD sequence module is shown in table 5b SEQ ID NO: 40.

In still another preferred embodiment of the invention, the fusion polypeptide comprises three soluble OX40L domains fused by peptide linker elements of SEQ ID NO: 2. The first soluble OX40L domain (i) consists of amino acids 56-183 of human OX40L according to SEQ ID NO: 1 with Y560 mutation and the soluble OX40L domains (iii) and (v) consist of amino acids 57-183 of human OX40L according to SEQ ID NO: 1 The resulting scOX40L-RBD sequence module is shown in table 5b SEQ ID NO: 41

In still another embodiment of the invention, the fusion polypeptide comprises three soluble OX40L domains fused by peptide linker elements of SEQ ID NO: 2. The first soluble OX40L domain (i) consists of amino acids 56-183 of human OX40L with Y56Q mutation according to SEQ ID NO: 1 and the soluble OX40L domains (iii) and (v) consist of amino acids 58-183 of human OX40L according to SEQ ID NO: 1. The resulting scOX40L-RBD sequence module is shown in table 5b SEQ ID NO: 42.

In a further preferred embodiment of the invention, the fusion polypeptide comprises three soluble OX40L domains fused by peptide linker elements of SEQ ID NO: 2. All three soluble OX40L domain (i), (iii), (v) consists of amino acids 56-183 of human OX40L according to SEQ ID NO: 1 with Y56G mutation. The resulting scOX40L-RBD sequence module is shown in table 5b SEQ ID NO: 43, which is well suited to generate fusion proteins with additional domains fused to either N-or C-terminal end with enhanced stability compared to wild type.

In another embodiment of the invention, the fusion polypeptide comprises three soluble OX40L domains fused by peptide linker elements of SEQ ID NO: 2. The first soluble OX40L domains (i) and (iii), consists of amino acids 55-183 of human OX40L according to SEQ ID NO: 1. The third soluble OX40L domain (v) is C-terminal shortened and consists of amino acids 55-179 with C97S mutation. The resulting scOX40L-RBD sequence module is shown in table 5b SEQ ID NO: 44.

Preferred Configuration OX40L-Fc

Additionally, the fusion polypeptide comprises an antibody Fc fragment domain according to SEQ ID NO: 13 that is fused C-terminally to the soluble OX40L domain (v) via a hinge-linker according to SEQ ID NO: 16. The inventors surprisingly found that this particular fusion polypeptide provides improved biological activity as compared to bivalent agonistic anti-OX40-mAB and has a prolonged stability as compared to fusion proteins comprising a lysine in position 223 and a N2975 mutation in the CH2 domain (according to the EU numbering). The amino acid sequence of an exemplary embodiment of an OX40 receptor agonist protein of the invention is set forth in SEQ ID NO: 27.

Further, the fusion polypeptide may comprise an N-terminal signal peptide domain e.g. according to SEQ ID NO: 17. A specific example of an OX40 receptor agonist protein of the invention is shown in SEQ ID NO: 25.

According to another preferred embodiment, the fusion polypeptide may additionally comprise a C-terminal Strep-tag that is fused to the polypeptide of the invention via a short serine linker as shown in SEQ ID NO: 18. According to this aspect of the invention, the Fc fragment preferably consists of the amino acid sequence as shown in SEQ ID NO: 13 or 14. Further, the Fc fragment may consist of a shorter Fc fragment, for example including amino acids 1-217 of SEQ ID NO: 13. Particularly preferred examples of fusion polypeptides comprising a C-terminal Strep-tag are shown in SEQ ID NO: 15 (PROTEIN A).

The exemplary OX40 receptor agonist proteins as shown in SEQ ID Nos: 15, 25, and 26, each comprises an N-terminal signal peptide domain, at amino acids 1-20 of each sequence. In each case, the mature protein starts with amino acid 21. Mature exemplary OX40 receptor agonist proteins (without a signal peptide) of the instant invention are set forth in SEQ ID NO: 27-34. Exemplary OX40 receptor agonist proteins described above are shown in Table 5.

The OX40 receptor agonist as set forth in SEQ ID NO: 27 has a reduced total number of glycosylation sites (the N2973 mutation in the CH2 region providing an aglycosylated CH2 domain, according to the EU numbering system), an increased number of inter-chain disulfide bonds in the hinge region, and the mutation of an upper-hinge lysine to a glycine (K223G, according to the EU numbering system). These alterations provide a decrease in potential degradation and OX40 receptor superclustering (along with concomitant toxicity).

According to one embodiment of the invention, the single-chain OX40L fusion polypeptide domain comprises three soluble OX40L domains fused by peptide linker elements of SEQ ID NO: 2. The soluble OX40L domains (i), (iii) and (v) each consists of amino acids 55-183 of human OX40L according to SEQ ID NO: 1 optionally with the soluble domains (i) (iii) and (v) comprising the Y56S mutation. A specific example of a single-chain-OX40L polypeptide comprising aforementioned OX40L Y565 muteins in domains (i), (iii) and (v) is shown in SEQ ID: 39 (Table 5B). In a preferred embodiment, an antibody Fc fragment domain according to SEQ ID NO: 13 is fused C-terminally to the soluble OX40L domain (v) of SEQ ID: 39 via a hinge linker according to SEQ ID NO: 16. A specific example of an OX40 receptor agonist protein of the invention comprising the SEQ ID NO: 39, the hinge linker of SEQ ID NO: 16 and an antibody Fc fragment according to SEQ ID NO: 13 is shown in SEQ ID NO: 30 (Table 5):

The OX40 receptor agonist as set forth in SEQ ID NO: 30 comprises the same layout as SEQ ID NO: 27 but with the Y565 mutation in the soluble OX40L domains (i), (iii) and (v) employing the scOX40L-RBD module shown SEQ ID NO: 39.

The OX40 receptor agonist as set forth in SEQ ID NO: 31 comprises the same layout as SEQ ID NO: 30 but with the second peptide linker (iv) shortened, thereby reducing promotor dissociation and enhancing the proteins stability towards proteases.

The OX40 receptor agonist as set forth in SEQ D NO: 32 comprises the same layout as SEQ ID NO:30 but with the third peptide linker (vi) shortened to reduce the interdomain distance between the soluble OX40L domain (v) and the Fc-domain (Vii) thereby enhancing the proteins stability towards proteases.

The OX40 receptor agonist as set forth in SEQ D NO: 33 comprises a scOX40L-RBD module with SEQ ID NO: 41, a third peptide linker with SEQ ID NO: 16 and (vii) an antibody Fc fragment with SEQ D NO: 13. The mature protein comprises the N-terminal Y56Q mutation thereby enabling formation of pyroglutamate leading to protection of the N-terminus against aminopeptidases and subsequently enhancing the overall stability of the protein during manufacture and storage,

The OX40 receptor agonist as set forth in SEQ ID NO: 34 comprises a scOX40L-RBD module with SEQ ID NO: 42, a third peptide linker with SEQ ID NO: 16 and (vii) an antibody Fc fragment with SEQ ID NO: 13,

The OX40 receptor agonist as set forth in SEQ ID NO: 35 comprises scOX40L-RBD module with SEQ ID NO: 44, a third peptide linker with SEQ ID NO: 16 and (vii) an antibody Fc fragment with SEQ ID NO: 13. This OX40 receptor agonist has a scOX40L-module with one OX40 receptor binding site mutated to not bind the OX40 receptor efficiently.

TABLE 5
Exemplary OX40 receptor agonist Proteins

| SEQ ID NO | Sequence |
|---|---|
| 25 | METDTLLVFVLLVWVPAGNGRYPRIQSIKVQFTEYKKEKGFILTSQKEDEIMKV |
| PROTEIN A | QNNSVIINCDGFYLISLKGYFSQEVNISLHYQKDEEPLFQLKKVRSVNSLMVAS |
| without | LTYKDKVYLNVTTDNTSLDDFHVNGGELILIHQNPGEFCVLGSGSGNGSRYPRI |
| StrepTag | QSIKVQFTEYKKEKGFILTSQKEDEIMKVQNNSVIINCDGFYLISLKGYFSQEV |
| | NISLHYQKDEEPLFQLKKVRSVNSLMVASLTYKDKVYLNVTTDNTSLDDFHVNG |
| | GELILIHQNPGEFCVLGSGSGNGSRYPRIQSIKVQFTEYKKEKGFILTSQKEDE |
| | IMKVQNNSVIINCDGFYLISLKGYFSQEVNISLHYQKDEEPLFQLKKVRSVNSL |
| | MVASLTYKDKVYLNVTTDNTSLDDFHVNGGELILIHQNPGEFCVLGSSSSSSSS |
| | GSCDKTHTCPPCPAPELLGGPSVFLFPPKPKDTLMISRTPEVTCVVVDVSHEDP |
| | EVKFNWYVDGVEVHNAKTKPREEQYSSTYRVVSVLTVLHQDWLNGKEYKCKVSN |
| | KALPAPIEKTISKAKGQPREPQVYTLPPSREEMTKNQVSLTCLVKGFYPSDIAV |
| | EWESNGQPENNYKTTPPVLDSDGSFFLYSKLTVDKSRWQQGNVFSCSVMHEALH |
| | NHYTQKSLSLSPGK |
| 15 | METDTLLVFVLLVWVPAGNGRYPRIQSIKVQFTEYKKEKGFILTSQKEDEIMKV |
| PROTEIN A | QNNSVIINCDGFYLISLKGYFSQEVNISLHYQKDEEPLFQLKKVRSVNSLMVAS |
| | LTYKDKVYLNVTTDNTSLDDFHVNGGELILIHQNPGEFCVLGSGSGNGSRYPRI |
| | QSIKVQFTEYKKEKGFILTSQKEDEIMKVQNNSVIINCDGFYLISLKGYFSQEV |
| | NISLHYQKDEEPLFQLKKVRSVNSLMVASLTYKDKVYLNVTTDNTSLDDFHVNG |
| | GELILIHQNPGEFCVLGSGSGNGSRYPRIQSIKVQFTEYKKEKGFILTSQKEDE |
| | IMKVQNNSVIINCDGFYLISLKGYFSQEVNISLHYQKDEEPLFQLKKVRSVNSL |
| | MVASLTYKDKVYLNVTTDNTSLDDFHVNGGELILIHQNPGEFCVLGSSSSSSSS |
| | GSCDKTHTCPPCPAPELLGGPSVFLFPPKPKDTLMISRTPEVTCVVVDVSHEDP |
| | EVKFNWYVDGVEVHNAKTKPREEQYSSTYRVVSVLTVLHQDWLNGKEYKCKVSN |
| | KALPAPIEKTISKAKGQPREPQVYTLPPSREEMTKNQVSLTCLVKGFYPSDIAV |
| | EWESNGQPENNYKTTPPVLDSDGSFFLYSKLTVDKSRWQQGNVFSCSVMHEALH |
| | NHYTQKSLSLSPGSSSSSSAWSHPQFEK |
| 26 | METDTLLVFVLLVWVPAGNGRYPRIQSIKVQFTEYKKEKGFILTSQKEDEIMKV |
| OX40L-wt + | QNNSVIINCDGFYLISLKGYFSQEVNISLHYQKDEEPLFQLKKVRSVNSLMVAS |
| SEQ14 | LTYKDKVYLNVTTDNTSLDDFHVNGGELILIHQNPGEFCVLGSGSGNGSRYPRI |
| | QSIKVQFTEYKKEKGFILTSQKEDEIMKVQNNSVIINCDGFYLISLKGYFSQEV |
| | NISLHYQKDEEPLFQLKKVRSVNSLMVASLTYKDKVYLNVTTDNTSLDDFHVNG |
| | GELILIHQNPGEFCVLGSGSGNGSRYPRIQSIKVQFTEYKKEKGFILTSQKEDE |
| | IMKVQNNSVIINCDGFYLISLKGYFSQEVNISLHYQKDEEPLFQLKKVRSVNSL |
| | MVASLTYKDKVYLNVTTDNTSLDDFHVNGGELILIHQNPGEFCVLGSSSSSSSS |
| | GSCDKTHTCPPCPAPPVAGPSVFLFPPKPKDTLMISRTPEVTCVVVDVSHEDPE |
| | VKFNWYVDGVEVHNAKTKPREEQYNSTYRVVSVLTVLHQDWLNGKEYKCKVSNK |
| | GLPSSIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHN |
| | HYTQKSLSLSPGK |
| 27 | RYPRIQSIKVQFTEYKKEKGFILTSQKEDEIMKVQNNSVIINCDGFYLISLKGY |
| OX40L- | FSQEVNISLHYQKDEEPLFQLKKVRSVNSLMVASLTYKDKVYLNVTTDNTSLDD |
| wt + SEQ13(FC) | FHVNGGELILIHQNPGEFCVLGSGSGNGSRYPRIQSIKVQFTEYKKEKGFILTS |
| No Signal | QKEDEIMKVQNNSVIINCDGFYLISLKGYFSQEVNISLHYQKDEEPLFQLKKVR |
| No Step | SVNSLMVASLTYKDKVYLNVTTDNTSLDDFHVNGGELILIHQNPGEFCVLGSGS |
| No Glyco | GNGSRYPRIQSIKVQFTEYKKEKGFILTSQKEDEIMKVQNNSVIINCDGFYLIS |
| | LKGYFSQEVNISLHYQKDEEPLFQLKKVRSVNSLMVASLTYKDKVYLNVTTDNT |
| | SLDDFHVNGGELILIHQNPGEFCVLGSSSSSSSSGSCDKTHTCPPCPAPELLGG |
| | PSVFLFPPKPKDTLMISRTPEVTCVVVDVSHEDPEVKFNWYVDGVEVHNAKTKP |
| | REEQYSSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGK |
| 28 | RYPRIQSIKVQFTEYKKEKGFILTSQKEDEIMKVQNNSVIINCDGFYLISLKGY |
| Deglyco-Fc | FSQEVNISLHYQKDEEPLFQLKKVRSVNSLMVASLTYKDKVYLNVTTDNTSLDD |
| No Signal | FHVNGGELILIHQNPGEFCVLGSGSGNGSRYPRIQSIKVQFTEYKKEKGFILTS |
| StrepTag | QKEDEIMKVQNNSVIINCDGFYLISLKGYFSQEVNISLHYQKDEEPLFQLKKVR |
| | SVNSLMVASLTYKDKVYLNVTTDNTSLDDFHVNGGELILIHQNPGEFCVLGSGS |
| | GNGSRYPRIQSIKVQFTEYKKEKGFILTSQKEDEIMKVQNNSVIINCDGFYLIS |
| | LKGYFSQEVNISLHYQKDEEPLFQLKKVRSVNSLMVASLTYKDKVYLNVTTDNT |
| | SLDDFHVNGGELILIHQNPGEFCVLGSSSSSSSSGSCDKTHTCPPCPAPELLGG |
| | PSVFLFPPKPKDTLMISRTPEVTCVVVDVSHEDPEVKFNWYVDGVEVHNAKTKP |
| | REEQYSSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGSSSSSSA |
| | WSHPQFEK |
| 29 | RYPRIQSIKVQFTEYKKEKGFILTSQKEDEIMKVQNNSVIINCDGFYLISLKGY |
| Glyco FC | FSQEVNISLHYQKDEEPLFQLKKVRSVNSLMVASLTYKDKVYLNVTTDNTSLDD |
| No Signal | FHVNGGELILIHQNPGEFCVLGSGSGNGSRYPRIQSIKVQFTEYKKEKGFILTS |
| No strep | QKEDEIMKVQNNSVIINCDGFYLISLKGYFSQEVNISLHYQKDEEPLFQLKKVR |
| | SVNSLMVASLTYKDKVYLNVTTDNTSLDDFHVNGGELILIHQNPGEFCVLGSGS |
| | GNGSRYPRIQSIKVQFTEYKKEKGFILTSQKEDEIMKVQNNSVIINCDGFYLIS |
| | LKGYFSQEVNISLHYQKDEEPLFQLKKVRSVNSLMVASLTYKDKVYLNVTTDNT |
| | SLDDFHVNGGELILIHQNPGEFCVLGSSSSSSSSGSCDKTHTCPPCPAPPVAGP |
| | SVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKGLPSSIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 30 | RsPRIQSIKVQFTEYKKEKGFILTSQKEDEIMKVQNNSVIINCDGFYLISLKGY |
| SEQ39 + FC13 | FSQEVNISLHYQKDEEPLFQLKKVRSVNSLMVASLTYKDKVYLNVTTDNTSLDD |
| | FHVNGGELILIHQNPGEFCVLGSGSGNGSRsPRIQSIKVQFTEYKKEKGFILTS |
| | QKEDEIMKVQNNSVIINCDGFYLISLKGYFSQEVNISLHYQKDEEPLFQLKKVR |
| | SVNSLMVASLTYKDKVYLNVTTDNTSLDDFHVNGGELILIHQNPGEFCVLGSGS |
| | GNGSRsPRIQSIKVQFTEYKKEKGFILTSQKEDEIMKVQNNSVIINCDGFYLIS |
| | LKGYFSQEVNISLHYQKDEEPLFQLKKVRSVNSLMVASLTYKDKVYLNVTTDNT |
| | SLDDFHVNGGELILIHQNPGEFCVLGSSSSSSSSGSCDKTHTCPPCPAPELLGG |
| | PSVFLFPPKPKDTLMISRTPEVTCVVVDVSHEDPEVKFNWYVDGVEVHNAKTKP |
| | REEQYSSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGK |
| 31 | RsPRIQSIKVQFTEYKKEKGFILTSQKEDEIMKVQNNSVIINCDGFYLISLKGY |
| | FSQEVNISLHYQKDEEPLFQLKKVRSVNSLMVASLTYKDKVYLNVTTDNTSLDD |
| | FHVNGGELILIHQNPGEFCVLGSGSGNGSRsPRIQSIKVQFTEYKKEKGFILTS |
| | QKEDEIMKVQNNSVIINCDGFYLISLKGYFSQEVNISLHYQKDEEPLFQLKKVR |
| | SVNSLMVASLTYKDKVYLNVTTDNTSLDDFHVNGGELILIHQNPGEFCVLGSGS |
| | RsPRIGSIKVQFTEYKKEKGFILTSQKEDEIMKVQNNSVIINCDGFYLISLKGY |
| | FSQEVNISLHYQKDEEPLFQLKKVRSVNSLMVASLTYKDKVYLNVTTDNTSLDD |
| | FHVNGGELILIHQNPGEFCVLGSSSSSSSSGSCDKTHTCPPCPAPELLGGPSVF |
| | LFPPKPKDTLMISRTPEVTCVVVDVSHEDPEVKFNWYVDGVEVHNAKTKPREEQ |
| | YSSTYRVVSVLTVLHQDWLNGKEYKCKVSNKALPAPIEKTISKAKGQPREPQVY |
| | TLPPSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |
| 32 | RsPRIQSIKVQFTEYKKEKGFILTSQKEDEIMKVQNNSVIINCDGFYLISLKGY |
| | FSQEVNISLHYQKDEEPLFQLKKVRSVNSLMVASLTYKDKVYLNVTTDNTSLDD |
| | FHVNGGELILIHQNPGEFCVLGSGSGNGSRsPRIQSIKVQFTEYKKEKGFILTS |
| | QKEDEIMKVQNNSVIINCDGFYLISLKGYFSQEVNISLHYQKDEEPLFQLKKVR |
| | SVNSLMVASLTYKDKVYLNVTTDNTSLDDFHVNGGELILIHQNPGEFCVLGSGS |
| | GNGSRsPRIQSIKVQFTEYKKEKGFILTSQKEDEIMKVQNNSVIINCDGFYLIS |
| | LKGYFSQEVNISLHYQKDEEPLFQLKKVRSVNSLMVASLTYKDKVYLNVTTDNT |
| | SLDDFHVNGGELILIHQNPGEFCVLGSSSSSGSCDKTHTCPPCPAPELLGGPSV |
| | FLFPPKPKDTLMISRTPEVTCVVVDVSHEDPEVKFNWYVDGVEVHNAKTKPREE |
| | QYSSTYRVVSVLTVLHQDWLNGKEYKCKVSNKALPAPIEKTISKAKGQPREPQV |
| | YTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK |
| 33 | QPRIQSIKVQFTEYKKKEKGFILTSQKEDEMKVQNNSVIINCDGFYLISLKGYF |
| | SQEVNISLHYQKDEEPLFQLKKVRSVNSLMVASLTYKDKVYLNVTTDNTSLDDF |
| | HVNGGELILIHQNPGEFCVLGSGSGNGSPRIQSIKVQFTEYKKEKGFILTSQKE |
| | DEIMKVQNNSVIINCDGFYLISLKGYFSQEVNISLHYQKDEEPLFQLKKVRSVN |
| | SLMVASLTYKDKVYLNVTTDNTSLDDFHVNGGELILIHQNPGEFCVLGSGSGNG |
| | SPRIQSIKVQFTEYKKEKGFILTSQKEDEIMKVQNNSVIINCDGFYLISLKGYF |
| | SQEVNISLHYQKDEEPLFQLKKVRSVNSLMVASLTYKDKVYLNVTTDNTSLDDF |
| | HVNGGELILIHQNPGEFCVLGSSSSSSSSGSCDKTHTCPPCPAPELLGGPSVFL |
| | FPPKPKDTLMISRTPEVTCVVVDVSHEDPEVKFNWYVDGVEVHNAKTKPREEQY |
| | SSTYRVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGK |
| 34 | QPRIQSIKVQFTEYKKEKGFILTSQKEDEIMKVQNNSVIINCDGFYLISLKGYF |
| | SQEVNISLHYQKDEEPLFQLKKVRSVNSLMVASLTYKDKVYLNVTTDNTSLDDF |
| | HVNGGELILIHQNPGEFCVLGSGSGNGSRIQSIKVQFTEYKKEKGFILTSQKED |
| | EIMKVQNNSVIINCDGFYLISLKGYFSQEVNISLHYQKDEEPLFQLKKVRSVNS |
| | LMVASLTYKDKVYLNVTTDNTSLDDFHVNGGELILIHQNPGEFCVLGSGSGNGS |
| | RIQSIKVQFTEYKKEKGFILTSQKEDEIMKVQNNSVIINCDGFYLISLKGYFSQ |
| | EVNISLHYQKDEEPLFQLKKVRSVNSLMVASLTYKDKVYLNVTTDNTSLDDFHV |
| | NGGELILIHQNPGEFCVLGSSSSSSSSGSCDKTHTCPPCPAPELLGGPSVFLFP |
| | PKPKDTLMISRTPEVTCVVVDVSHEDPEVKFNWYVDGVEVHNAKTKPREEQYSS |
| | TYRVVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGSFFL |
| | YSKLTVDKSRWQQGNVFSCSVMHEALHNHYTQKSLSLSPGK |
| 35 | RYPRIQSIKVQFTEYKKEKGFILTSQKEDEIMKVQNNSVIINCDGFYLISLKGY |
| | FSQEVNISLHYQKDEEPLFQLKKVRSVNSLMVASLTYKDKVYLNVTTDNTSLDD |
| | FHVNGGELILIHQNPGEFCVLGSGSGNGSRYPRIQSIKVQFTEYKKEKGFILTS |
| | QKEDEIMKVQNNSVIINCDGFYLISLKGYFSQEVNISLHYQKDEEPLFQLKKVR |
| | SVNSLMVASLTYKDKVYLNVTTDNTSLDDFHVNGGELILIHQNPGEFCVLGSGS |
| | GNGSRYPRIQSIKVQFTEYKKEKGFILTSQKEDEIMKVQNNSVIINsDGFYLIS |
| | LKGYFSQEVNISLHYQKDEEPLFQLKKVRSVNSLMVASLTYKDKVYLNVTTDNT |
| | SLDDFHVNGGELILIHQNPGEGSSSSSSSSGSCDKTHTCPPCPAPELLGGPSVF |
| | LFPPKPKDTLMISRTPEVTCVVVDVSHEDPEVKFNWYVDGVEVHNAKTKPREEQ |
| | YSSTYRVVSVLTVLHQDWLNGKEYKCKVSNKALPAPIEKTISKAKGQPREPQVY |
| | TLPPSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |

TABLE 5B
Exemplary scOX40L-RBD modules

| SEQ | |
|---|---|
| ID | |
| NO | Sequence |
| 36 | RYPRIQSIKVQFTEYKKEKGFILTSQKEDEIMKVQNNSVIINCDGFY |
| | LISLKGYFSQEVNISLHYQKDEEPLFQLKKVRSVNSLMVASLTYKDK |
| | VYLNVTTDNTSLDDFHVNGGELILIHQNPGEFCVLGSGSGNGSRYPR |
| | IQSIKVQFTEYKKEKGFILTSQKEDEIMKVQNNSVIINCDGFYLISL |
| | KGYFSQEVNISLHYQKDEEPLFQLFFVRSVNSLMVASLTYKDKVYLN |
| | VTTDNTSLDDFHVNGGELILIHQNPGEFCVLGSGSGNGSRYPRIQSI |
| | KVQFTEYKKEKGFILTSQKEDEIMKVQNNSVIINCDGFYLISLKGYF |
| | SQEVNISLHYQKDEEPLFQLKKVRSVNSLMVASLTYKDKVYLNVTTD |
| | NTSLDDFHVNGGELILIHQNPGEFCVL |
| 39 | RsPRIQSIKVQFTEYKKEKGFILTSQKEDEIMKVQNNSVIINCDGFY |
| | LISLKGYFSQEVNISLHYQKDEEPLFQLKKVRSVNSLMVASLTYKDK |
| | VYLNVTTDNTSLDDFHVNGGELILIHQNPGEFCVLGSGSGNGSRsPR |
| | IQSIKVQFTEYKKEKGFILTSQKEDEIMKVQNNSVIINCDGFYLISL |
| | KGYFSQEVNISLHYQKDEEPLFQLKKVRSVNSLMVASLTYKDKVYLN |
| | VTTDNTSLDDFHVNGGELILIHQNPGEFCVLGSGSGNGSRsPRIQSI |
| | KVQFTEYKKEKGFILTSQKEDEIMKVQNNSVIINCDGFYLISLKGYF |
| | SQEVNISLHYQKDEEPLFQLKKVRSVNSLMVASLTYKDKVYLNVTTD |
| | NTSLDDFHVNGGELILIHQNPGEFCVL |
| 40 | RYPRIQSIKVQFTEYKKEKGFILTSQKEDEIMKVQNNSVIINCDGFY |
| | LISLKGYFSQEVNISLHYQKDEEPLFQLKKVRSVNSLMVASLTYKDK |
| | VYLNVTTDNTSLDDFHVNGGELILIHQNPGEFCVLGSGSGNGSPRIQ |
| | SIKVQFTEYKKEKGFILTSQKEDEIMKVQNNSVIINCDGFYLISLKG |
| | YFSQEVNISLHYQKDEEPLFQLKKVRSVNSLMVASLTYKDKVYLNVT |
| | TDNTSLDDFHVNGGELILIHQNPGEFCVLGSGSGNGSPRIQSIKVQF |
| | TEYKKEKGFILTSQKEDEIMKVQNNSVIINCDGFYLISLKGYFSQEV |
| | NISLHYQKDEEPLFQLKKVRSVNSLMVASLTYKDKVYLNVTTDNTSL |
| | DDFHVNGGELILIHQNPGEFCVL |
| 41 | QPRIQSIKVQFTEYKKEKGFILTSQKEDEIMKVQNNSVIINCDGFYL |
| | ISLKGYFSQEVNISLHYQKDEEPLFQLKKVRSVNSLMVASLTYKDKV |
| | YLNVTTDNTSLDDFHVNGGELILIHQNPGEFCVLGSGSGNGSPRIQS |
| | IKVQFTEYKKEKGFILTSQKEDEIMKVQNNSVIINCDGFYLISLKGY |
| | FSQEVNISLHYQKDEEPLFQLKKVRSVNSLMVASLTYKDKVYLNVTT |
| | DNTSLDDFHVNGGELILIHQNPGEFCVLGSGSGNSPRIQSIKVQFTE |
| | GYKKEKGFILTSQKEDEIMKVQNNSVIINCDGFYLISLKGYFSQEVN |
| | ISLHYQKDEEPLFQLKKVRSVNSLMVASLTYKDKVYLNVTTDNTSLD |
| | DFHVNGGELILIHQNPGEFCVL |
| 42 | QPRIQSIKVQFTEYKKEKGFILTSQKEDEIMKVQNNSVIINCDGFYL |
| | ISLKGYFSQEVNISLHYQKDEEPLFQLKKVRSVNSLMVASLTYKDKV |
| | YLNVTTDNTSLDDFHVNGGELILIHQNPGEFCVLGSGSGNGSRIQSI |
| | KVQFTEYKKEKGFILTSQKEDEIMKVQNNSVIINCDGFYLISLKGYF |
| | SQEVNISLHYQKDEEPLFQLKKVRSVNSLMVASLTYKDKVYLNVTTD |
| | NTSLDDFHVNGGELILIHQNPGEFCVLGSGSGNGSRIQSIKVQFTEY |
| | KKEKGFILTSQKEDEIMKVQNNSVIINCDGFYLISLKGYFSQEVNIS |
| | LHYQKDEEPLFQLKKVRSVNSLMVASLTYKDKVYLNVTTDNTSLDDF |
| | HVNGGELILIHQNPGEFCVL |
| 43 | GPRIQSIKVQFTEYKKEKGFILTSQKEDEIMKVQNNSVIINCDGFYL |
| | ISLKGYFSQEVNISLHYQKDEEPLFQLKKVRSVNSLMVASLTYKDKV |
| | YLNVTTDNTSLDDFHVNGGELILIHQNPGEFCVLGSGSGNGSGPRIQ |
| | SIKVQFTEYKKEKGFILTSQKEDEIMKVQNNSVIINCDGFYLISLKG |
| | YFSQEVNISLHYQKDEEPLFQLKKVRSVNSLMVASLTYKDKVYLNVT |
| | TDNTSLDDFHVNGGELILIHQNPGEFCVLGSGSGNGSGPRIQSIKVQ |
| | FTEYKKEKGFILTSQKEDEIMKVQNNSVIINCDGFYLISLKGYFSQE |
| | VNISLHYQKDEEPLFQLKKVRSVNSLMVASLTYKDKVYLNVTTDNTS |
| | LDDFHVNGGELILIHQNPGEFCVL |
| 44 | RYPRIQSIKVQFTEYKKEKGFILTSQKEDEIMKVQNNSVIINCDGFY |
| | LISLKGYFSQEVNISLHYQKDEEPLFQLKKVRSVNSLMVASLTYKDK |
| | VYLNVTTDNTSLDDFHVNGGELILIHQNPGEFCVLGSGSGNGSRYPR |
| | IQSIKVQFTEYKKEKGFILTSQKEDEIMKVQNNSVIINCDGFYLISL |
| | SQKGYFEVNISLHYQKDEEPLFQLKKVRSVNSLMVASLTYKDKVYLN |
| | VTTDNTSLDDFHVNGGELILIHQNPGEFCVLGSGSGNGSRYPRIQSI |
| | KVQFTEYKKEKGFILTSQKEDEIMKVQNNSVIINsDGFYLISLKGYF |
| | SQEVNISHYLQKDEEPLFQLKKVRSVNSLMVASLTYKDKVYLNVTTD |
| | VNGNTSLDDFHGELILIHQNPGE |

Furthermore, it has to be noted that the scOX40L-RBD modules of Table 5B are well suited to generate fusion proteins with additional domains fused to either N-or C-terminal end employing the linkers described in Table 2 (SEQ ID NO: 2-12).

A further aspect of the present invention relates to a nucleic acid molecule encoding a OX40 receptor agonist protein as described herein. The nucleic acid molecule may be a DNA molecule, e.g. a double-stranded or single-stranded DNA molecule, or an RNA molecule. The nucleic acid molecule may encode the OX40 receptor agonist protein or a precursor thereof, e.g., a pro- or pre-proform of the OX40 receptor agonist protein which may comprise a signal sequence or other heterologous amino acid portions for secretion or purification which are preferably located at the N- and/or C-terminus of the OX40 receptor agonist protein. The heterologous amino acid portions may be linked to the first and/or second domain via a protease cleavage site, e.g. a Factor X3, thrombin or IgA protease cleavage site. A specific example of a nucleic acid sequence of the invention is shown in Table 6 as SEQ ID NO: 37. This nucleic acid molecule comprises the open reading frame encoding the fusion polypeptide of SEQ ID NO: 25.

TABLE 6
Nucleic Acid Sequence of Exemplary OX40 receptor
agonist Protein

| SEQ | |
|---|---|
| ID | |
| NO | Sequence |
| 37 | AAGCTTTAGGGATAACAGGGTAATAGCCGCCACCATGGAGACTGA |
| | CACCCTGCTGGTGTTCGTGCTGCTGGTCTGGGTGCCTGCAGGAAA |
| | TGGAAGGTATCCCAGGATTCAAAGCATCAAGGTGCAGTTCACAGA |
| | ATATAAGAAGGAGAAGGGATTTATCCTGACCAGCCAAAAGGAGGA |
| | CGAGATCATGAAAGTGCAAAATAACAGCGTCATCATTAATTGCGA |
| | CGGCTTCTACCTCATCTCCCTGAAGGGCTATTTTTCCCAAGAGGT |
| | GAACATCTCCCTGCACTACCAAAAAGACGAGGAGCCCCTCTTCAT |
| | ACTGAAGAAAGTGCGGTCCGTGAACTCCCTGATGGTGGCTTCCCT |
| | GACCTATAAGGACAAAGTGTATCTGAATGTGACCACCGATAACAC |
| | CTCCCTGGATGATTTCCATGTGAACGGAGGCGAACTGATCCTGAT |
| | CCACCAGAACCCTGGCGAATTTTGCGTGCTGGGCTCCGGATCTGG |
| | TAACGGTTCTCGGTACCCCAGGATTCAGTCCATTAAGGTCCAATT |
| | CACCGAGTACAAGAAAGAGAAGGGCTTCATCCTCACCTCCCAAAA |
| | GGAAGATGAGATTATGAAGGTGCAGAATAATAGCGTCATTATTAA |
| | TTGTGACGGATTCTATCTGATCTCCCTGAAAGGCTATTTCAGCCA |
| | GGAGGTGAATATCTCCCTGCATTACCAAAAAGATGAGGAGCCTCT |
| | CTTCCAGCTGAAAAAAGTGAGGTCCGTGAATTCCCTGATGGTGGC |
| | CTCCCTGACCTACAAAGATAAGGTGTATCTGAACGTGACCACCGA |
| | CAACACAAGCCTGGATGACTTCCACGTGAATGGAGGAGAGCTGAT |
| | CCTGATTCACCAGAATCCCGGAGAGTTTTGCGTCCTGGGCAGCGG |
| | TTCTGGTAACGGCTCTAGATATCCCCGTATTCAAAGCATCAAAGT |
| | CCAGTTTACCGAGTACAAAAAGGAGAAAGGATTCATCCTGACCAG |
| | CCAGAAAGAAGACGAGATTATGAAAGTGCGAACAATAGCGTCATA |
| | CATCAACTGCGATGGCTTTTACCTGATTAGCCTGAAGGGCTACTT |
| | TAGCCAGGAAGTGAATATCAGCCTGCATTATCAGAAGGACGAAGA |
| | ACCTCTCTTTCAGCTGAAAAAGGTGCGGAGCGTGAACAGCCTCAT |
| | GGTGGCCAGCCTGACCTATAAAGACAAGGTGTACCTGAATGTCAC |
| | CACCGATAATACCTCCCTGGACGACTTTCATGTGAATGGAGGCGA |
| | TCACTGATCCTGATCCAAAAATCCCGGCGAATTTTGCGTCCTGGG |
| | ATCCTCGAGTTCATCGTCCTCATCCGGCTCATGTGATAAGACCCA |
| | CACCTGCCCTCCCTGTCCTGCCCCTGAGCTGCTGGGCGGACCTTC |
| | TGTGTTCCTGTTCCCCCCCAAGCCTAAGGACACCCTGATGATCTC |
| | CAGGACCCCTGAGGTGACCTGTGTGGTGGTGGACGTGTCTCACGA |
| | AGATCCCGAGGTGAAGTTCAACTGGTACGTGGACGGCGTGGAGGT |
| | CCACAACGCCAAGACCAAGCCTAGGGAGGAGCAGTACAGCTCCAC |
| | CTACCGGGTGGTGTCTGTGCTGACCGTGCTGCACCAGGATTGGCT |
| | GAACGGAAAGGAGTATAAGTGTAAGGTCTCCAACAAGGCCCTGCC |
| | TGCCCCCATCGAGAAAACCATCTCCAAGGCCAAGGGCCAGCCTCG |
| | GGAGCCTCAGGTGTACACCCTGCCTCCTAGCAGGGAGGAGATGAC |
| | CAAGAACCAGGTGTCCCTGACCTGTCTGGTGAAGGGCTTCTACCC |
| | TTCCGATATCGCCGTGGAGTGGGAGTCTAATGGCCAGCCCGAGAA |
| | AACCTACAAGACCACCCCTCCTGTGCTGGACTCTGACGGCTCCTT |
| | CTTCCTGTACTCCAAGCTGACCGTGGACAAGTCCAGATGGCAGCA |
| | CAGGGACGTGTTCTCCTGCTCCGTGATGCACGAGGCCCTGCACAA |
| | TCACTACACCCAGAAGTCCCTGTCTCTGAGTCCGGGCAAGTAATA |
| | GGCGCGCC |

The nucleic acid molecule may be operatively linked to an expression control sequence, e.g. an expression control sequence which allows expression of the nucleic acid molecule in a desired host cell. The nucleic acid molecule may be located on a vector, e,g. a plasmid, a bacteriophage, a viral vector, a chromosomal integration vector, etc. Examples of suitable expression control sequences and vectors are described for example by Sambrook et al. (1989) Molecular Cloning, A Laboratory Manual, Cold Spring Harbor Press, and Ausubel et al. (1989), Current Protocols in Molecular Biology, John Wiley & Sons or more recent editions thereof.

Various expression vector/host cell systems may be used to express the nucleic acid sequences encoding the OX40 receptor agonist proteins of the present invention. Suitable host cells include, but are not limited to, prokaryotic cells such as bacteria, e.g. E. coli, eukaryotic host cells such as yeast cells, insect cells, plant cells or animal cells, preferably mammalian cells and, more preferably, human cells. Further, the invention relates to a non-human organism transformed or transfected with a nucleic acid molecule as described above. Such transgenic organisms may be generated by known methods of genetic transfer including homologous recombination.

A further aspect of the present invention relates to a pharmaceutical or diagnostic composition comprising as the active agent at least one OX40 receptor agonist protein, a respective nucleic acid encoding therefore, or a transformed or transfected cell, all as described herein.

In another aspect, the present invention provides a pharmaceutical composition comprising an OX40 receptor agonist protein disclosed herein and one or more pharmaceutically acceptable carriers, diluents, excipients, and/or adjuvants. In another aspect, the present invention provides a nucleic acid molecule encoding the OX40 receptor agonist protein. In another embodiment, the present invention provides an expression vector comprising the nucleic acid molecule. In another embodiment, the present invention provides a cell comprising the nucleic acid molecule. In a further embodiment, the cell is a eukaryotic cell. In another embodiment, the cell is a mammalian cell. In another embodiment, the cell is a Chinese Hamster Ovary (CHO) cell. In other embodiments, the cell is selected from the group consisting of CHO-DBX11, CHO-DG44, CHO-S, and CHO-K1 cells. In other embodiments, the cell is selected from the group consisting of Vero, BHK, HeLa, COS, MDCK, HEK-293, NIH-3T3, W138, BT483, Hs578T, HTB2, BT20, T47D, NSO, CRL7030, HsS78Bst, PER.C6, SP2/0-Agl4, and hybridoma cells.

In another aspect, the present invention provides a method of treating a subject having an OX40L-associated disease or disorder, the method comprising administering to the subject an effective amount of the OX40 receptor agonist protein. In one embodiment, the OX40 receptor agonist protein is administered alone. In another embodiment, the OX40 receptor agonist protein is administered before, concurrently, or after the administration of a second agent. In another embodiment, the disease or disorder is selected from the group consisting of: tumors, infectious diseases, inflammatory diseases, metabolic diseases, autoimmune disorders, degenerative diseases, apoptosis-associated diseases, and transplant rejections. In one embodiment, the tumors are solid tumors. In one embodiment, the tumors arise from the group of cancers consisting of sarcoma, esophageal cancer, and gastric cancer. In another embodiment, the tumors arise from Ewing's sarcoma or fibrosarcoma. In another embodiment, the tumors arise from the group of cancers consisting of Non-Small Cell Lung Carcinoma (NSCLC), pancreatic cancer, colorectal cancer, breast cancer, ovarian cancer, head and neck cancers, and Small Cell Lung Cancer (SCLC). In another embodiment, the tumors are lymphatic tumors. In one embodiment, the tumors are hematologic tumors. In another embodiment, the tumors arise from non-Hodgkin's lymphoma, leukemia, acute lymphoblastic leukemia (ALL), acute myeloid leukemia (AML), B cell lymphoma, Burkitt's lymphoma, chronic myelocytic leukemia (CML), chronic lymphocytic leukemia (CLL), or hairy cell leukemia. In another embodiment, the autoimmune disorders are rheumatoid diseases, arthritic diseases, or rheumatoid and arthritic diseases. In a further embodiment, the disease or disorder is rheumatoid arthritis. In another embodiment, the degenerative disease is a neurodegenerative disease. In a further embodiment, the neurodegenerative disease is multiple sclerosis.

In one embodiment, the second agent is a chemotherapeutic, radiotherapeutic, or biological agent. In one embodiment, the second agent is selected from the group consisting of Duvelisib, Ibrutinib, Navitoclax, and Venetoclax. In another embodiment, the second agent is an apoptotic agent. In one embodiment, the apoptotic second agent is selected from the group consisting of Bortezomib, Azacitidine, Dasatinib, and Gefitinib. In a particular embodiment, the pharmaceutical compositions disclosed herein are administered to a patient by intravenous or subcutaneous administration. In other embodiments, the disclosed pharmaceutical compositions are administered to a patient byoral, parenteral, intramuscular, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal administration.

In one embodiment, the OX40 receptor agonist protein is administered as a single bolus. In another embodiment, OX40 receptor agonist protein may be administered over several divided doses. The OX40 receptor agonist protein can be administered at about 0.1-100 mg/kg. In one embodiment, the OX40 receptor agonist protein can be administered at a dosage selected from the group consisting of: about 0.1-0.5, 0.1-1, 0.1-10, 0,1-20, 0.1-50, 0.1-75, 1-10, 1-15, 1-7.5, 1.25-15, 1.25-7.5, 2.5-7.5, 2.5-15, 5-15, 5-7.5,1-20, 1-50, 7-75, 1-100, 5-10, 5-15, 5-20, 5-25, 5-50, 5-75, 10-20, 10-50, 10-75, and 10-100 mg/kg. In other embodiments, the OX40 receptor agonist protein is present in pharmaceutical compositions at about 0.1-100 mg/ml. In one embodiment, the OX40 receptor agonist protein is present in pharmaceutical compositions at an amount selected from the group consisting of: about 0.1-0.5, 0.1-1, 0.1-10, 0.1-20, 0.1-50, 0.1-75, 1-10, 1-20, 1-50, 1-75, 1-100, 5-10, 5-15, 5-20, 5-25, 5-50, 5-75, 10-20, 10-50, 10-75, or 10-100 mg/ml. In other embodiments, a therapeutically effective amount of OX40 receptor agonist protein is administered to a subject. In another embodiment, a prophylactically effective amount of OX40 receptor agonist protein is administered to a subject.

The term “OX40L-associated disease or disorder” as used herein is any disease or disorder which may be ameliorated by administering an effective amount of an OX40 receptor agonist to a subject in need thereof. At least one OX40 receptor agonist protein, respective nucleic acid encoding therefore, or transformed or transfected cell, all as described herein may be used in therapy, e.g., in the prophylaxis and/or treatment of disorders caused by, associated with and/or accompanied by dysfunction of OX40L, particularly proliferative disorders, such as tumors, e.g., solid or lymphatic tumors; infectious diseases; inflammatory diseases; metabolic diseases; autoimmune disorders, e,g. rheumatoid and/or arthritic diseases; degenerative diseases, e,g. neurodegenerative diseases such as multiple sclerosis; apoptosis-associated diseases or transplant rejections.

The term “dysfunction of OX40L” as used herein is to be understood as any function or expression of OX40L that deviates from the normal function or expression of OX40L, e.g., overexpression of the OX40L gene or protein, reduced or abolished expression of the OX40L gene or protein compared to the normal physiological expression level of OX40L, increased activity of OX40L, reduced or abolished activity of OX40L, increased binding of OX40L to any binding partners, e,g., to a receptor, particularly a OX40L receptor or another cytokine molecule, reduced or abolished binding to any binding partner, e.g. to a receptor, particularly a OX40L receptor or another cytokine molecule, compared to the normal physiological activity or binding of OX40L.

In various embodiments, a method is provided for diagnosing and/or treating a human subject suffering from a disorder which can be diagnosed and/or reated by targeting OX40L receptors comprising administering to the human subject a OX40 receptor agonist protein disclosed herein such that the effect on the activity of the target, or targets, in the human subject is agonistic, one or more symptoms is alleviated, and/or treatment is achieved. The OX40 receptor agonist proteins provided herein can be used to diagnose and/or treat humans suffering from primary and metastatic cancers, including carcinomas of breast, colon, rectum, lung (e.g., small cell lung cancer “SCLC” and non-small cell lung cancer “NSCLC”), oropharynx, hypopharynx, esophagus, stomach, pancreas, liver, gallbladder and bile ducts, small intestine, urinary tract (including kidney, bladder and urothelium), female genital tract (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma), tumors of the brain, nerves, eyes, and meninges (including astrocytomas, gliomas, glioblastomas, retinoblastomas, neuromas, neuroblastomas, Schwannomas, and meningiomas), tumors arising from hematopoietic malignancies, acute leukemia, acute lymphoblastic leukemia (ALL), acute myeloid leukemia (AML), B cell lymphoma, Burkitt's lymphoma, chronic myelocytic leukemia (CML), chronic lymphocytic leukemia (CLL), hairy cell leukemia, Hodgkin's and non-Hodgkin's lymphomas, DLBCL, follicular lymphomas, hematopoietic malignancies, Kaposi's sarcoma, malignant lymphoma, malignant histiocytosis, malignant melanoma, multiple myeloma, paraneoplastic syndrome/hypercalcemia of malignancy, or solid tumors.

A pharmaceutical composition comprising an OX40 receptor agonist protein disclosed herein and a pharmaceutically acceptable carrier is provided. In some embodiments, the pharmaceutical composition comprises at least one additional therapeutic agent for treating a disorder. For example, the additional agent may be a therapeutic agent, a chemotherapeutic agent; an imaging agent, a cytotoxic agent, an angiogenesis inhibitor, a kinase inhibitor (including but not limited to a KDR and a TIE-2 inhibitor), a co-stimulation molecule modulator or an immune checkpoint inhibitor (including but not limited to anti-B7.1, anti-B7.2, anti-B7.3, anti-B7.4, anti-CD28, anti-B7RP1, CTLA4-Ig, anti-CTLA-4, anti-PD-1, anti-PD-L1, anti-PD-L2, anti-ICOS, anti-LAG-3, anti-Tim3, anti-VISTA, anti-HVEM, anti-BTLA, LIGHT fusion protein, anti-CD137, anti-CD137L, anti-OX40, anti-OX40L, anti-CD70, anti-CD27, anti-CD27L, anti-GALS, anti-AZAR, anti-KIR, anti-IDO-1, anti-CD20), a dendritic cell/antigen-presenting cell modulator (including but not limited to anti-CD40 antibody, anti-CD4OL, anti-DC-SIGN, anti-lectin-1, anti-CD301, anti-CD303, anti-CD123, anti-CD267, anti-DNGR1, anti-CD265, anti-DCIR, anti-CD266, anti-ILT7), a modulator for Toll-like receptors (including but not limited to anti-TLR-1, anti-TLR-2, anti-TLR-3, anti-TLR-4, anti-TLR-4, anti-TLR-5, anti-TLR-6, anti-TLR-7, anti-TLR-8, anti-TLR-9), an adhesion molecule blocker (including but not limited to an anti-LFA-1 antibody, an anti-E/L selectin antibody, a small molecule inhibitor), an anti-cytokine antibody or functional fragment thereof (including but not limited to an anti-IL-18, an anti-TNF, or an anti-IL-6/cytokine receptor antibody), a bispecific redirected T cell or NK cell cytotoxicity (including but not limited to a BITE®), a chimeric T cell receptor (CAR-T) based therapy, a T cell receptor (TCR)-based therapy, a therapeutic cancer vaccine, methotrexate, cyclosporin, rapamycin, FK506, a detectable label or reporter, a TNF antagonist, an anti-rheumatic, a muscle relaxant, a narcotic, a non-steroid anti-inflammatory drug (NSAID), an analgesic, an anesthetic, a sedative, a local anesthetic, a neuromuscular blocker, an antimicrobial, an antipsoriatic, a corticosteriod, an anabolic steroid, an erythropoietin, an immunization, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, or a cytokine antagonist.

In an embodiment, a method of treating a cancer or in the prevention or inhibition of metastases from the tumors described herein, the OX40 receptor agonist protein(s) can be used alone or in combination with one or more additional agents, e.g., a chemotherapeutic, radiotherapy, or biological agent. In some embodiments, the agent can include the following:13-cis-Retinoic Acid; 2-CdA; 2-Chlorodeoxyadenosine; 5-Azacitidine; 5-Fluorouracil; 6-Mercaptopurine; 6-MP; 6-TG; 6-Thioguanine; Abraxane; Accutane®; Actinomycin-D; Adriamycin®; Adrucil®; Afinitor®; Agrylin®; Ala-Cort®; Aldesleukin; Alemtuzumab; ALIMTA; Alitretinoin; Alkaban-AQ®; Alkeran®; All-transretinoic Acid; Alpha Interferon; Altretamine; Amethopterin; Amifostine; Aminoglutethimide; Anagrelide; Anandron®; Anastrozole; Arabinosylcytosine; Ara-C Aranesp®; Aredia®; Arimidex®; Aromasin®; Arranon®; Arsenic Trioxide; Arzerra™; Asparaginase; ATRA; Avastin®; Azacitidine; BCG; BCNU; Bendamustine; Bevacizumab; Bexarotene; BEXXAR®; Bicalutamide; BiCNU; Blenoxane® Bleomycin; Bortezomib; Busulfan; Busulfex®; C225; Calcium Leucovorin; Campath®; Camptosar®; Camptothecin-11; Capecitabine Carac™; Carboplatin; Carmustine; Carmustine Wafer; Casodex®; CC-5013; CCI-779; CCNU; CDDP; CeeNU; Cerubidine®; Cetuximab; Chlorambucil; Cisplatin; Citrovorum Factor; Cladribine; Cortisone; Cosmegen®; CPT-11; Cyclophosphamide; Cytadren®; Cytarabine; Cytarabine Liposomal; Cytosar-U®; Cytoxan®; Dacarbazine; Dacogen; Dactinomycin; Darbepoetin Alfa; Dasatinib; Daunomycin; Daunorubicin; Daunorubicin Hydrochloride; Daunorubicin Liposomal; DaunoXome®; Decadron; Decitabine; Delta-Cortef®; Deltasone®; Denileukin; Diftitox; DepoCyt™; Dexamethasone; Dexamethasone Acetate; Dexamethasone Sodium Phosphate; Dexasone; Dexrazoxane; DHAD; DIC; Diodex; Docetaxel; Doxil®; Doxorubicin; Doxorubicin Liposomal; Droxia™; DTIC; DTIC-Dome®; Duralone®; Duvelisib; Efudex®; Eligard™; Ellence™; Eloxatin™; Elspar®; Emcyt®; Epirubicin; Epoetin Alfa; Erbitux; Erlotinib; Erwinia L-asparaginase; Estramustine; Ethyol Etopophos®; Etoposide; Etoposide Phosphate; Eulexin®; Everolimus; Evista®; Exemestane; Fareston®; Faslodex®; Ferrara®; Filgrastim; Floxuridine; Fludara®; Fludarabine; Fluoroplex®; Fluorouracil; Fluorouracil (cream); Fluoxymesterone; Flutamide; Folinic Acid; FUDR®; Fulvestrant; Gefitinib; Gemcitabine; Gemtuzumab ozogamicin; Gemzar; Gleevec™; Gliadel® Wafer; GM-CSF; Goserelin; Granulocyte-Colony Stimulating Factor (G-CSF); Granulocyte Macrophage Colony Stimulating Factor (G-MCSF); Halotestin®; Herceptin®; Hexadrol; Hexalen®; Hexamethylmelamine; HMM; Hycamtin®; Hydrea®; Hydrocort Acetate®; Hydrocortisone; Hydrocortisone Sodium Phosphate; Hydrocortisone Sodium Succinate; Hydrocortone Phosphate; Hydroxyurea; Ibrutinib; Ibritumomab; Ibritumomab Tiuxetan; Idamycin®; Idarubicin Ifex®; Interferon-alpha; Interferon-alpha-2b (PEG Conjugate); Ifosfamide; Interleukin-11 (IL-11); Interleukin-2 (IL-2); Imatinib mesylate; Imidazole Carboxamide; Intron A®; ipilimumab, Iressa®; Irinotecan; Isotretinoin; Ixabepilone; Ixempra™; KADCYCLA®; Kidrolase (t) Lanacort®; Lapatinib; L-asparaginase; LCR; Lenalidomide; Letrozole; Leucovorin; Leukeran; Leukine™; Leuprolide; Leurocristine; Leustatin™; Lirilumab; Liposomal Ara-C; Liquid Pred®; Lomustine; L-PAM; L-Sarcolysin; Lupron®; Lupron Depot®; Matulane®; Maxidex; Mechlorethamine; Mechlorethamine Hydrochloride; Medralone®; Medrol®; Megace®; Megestrol; Megestrol Acetate; MEK inhibitors; Melphalan; Mercaptopurine; Mesna; Mesnex™; Methotrexate; Methotrexate Sodium; Methylprednisolone; Meticorten®; Mitomycin; Mitomycin-C; Mitoxantrone M-Prednisol®; MTC; MTX; Mustargen®; Mustine; Mutamycin®; Myleran®; Mylocel™; Mylotarg®; Navitoclax; Navelbine®; Nelarabine; Neosar®; Neulasta™; Neumega®; Neupogen®; Nexavar®; Nilandron®; Nilotinib; Nilutamide; Nipent®; Nitrogen Mustard Novaldex®; Nivolumab; Novantrone®; Nplate; Octreotide; Octreotide acetate; Ofatumumab; Oncospar®; Oncovin®; Ontak®; Onxal™; Oprelvekin; Orapred®; Orasone®; Oxaliplatin; Paclitaxel; Paclitaxel Protein-bound; Pamidronate; Panitumumab; Panretin®; Paraplatin®; Pazopanib; Pediapred®; PEG Interferon; Pegaspargase; Pegfilgrastim; PEG-INTRONT™; PEG-L-asparaginase; PEMETREXED; Pembrolizumab; Pentostatin; Pertuzumab; Phenylalanine Mustard; Pidilizumab; Platinol®; Platinol-AQ®; Prednisolone; Prednisone; Prelone®; Procarbazine; PROCRIT®; Proleukin®; Prolifeprospan 20 with Carmustine Implant; Purinethol®; BRAF inhibitors; Raloxifene; Revlimid®; Rheumatrex®; Rituxan®; Rituximab; Roferon-A®; Romiplostim; Rubex®; Rubidomycin hydrochloride; Sandostatin® Sandostatin LAR®; Sargramostim; Solu-Cortef®; Solu-Medrol®; Sorafenib; SPRYCEL™; STI-571; STIVAGRA™, Streptozocin; SU11248; Sunitinib; Sutent®; Tamoxifen Tarceva®; Targretin®; Tasigna®; Taxol®; Taxotere®; Temodar®; Temozolomide Temsirolimus; Teniposide; TESPA; Thalidomide; Thalomid®; TheraCys®; Thioguanine; Thioguanine Tabloid®; Thiophosphoamide; Thioplex®; Thiotepa; TICE®; Toposar®; Topotecan; Toremifene; Torisel®; Tositumomab; Trastuzumab; Treanda®; Tremelimumab; Tretinoin; Trexall™; Trisenox®; TSPA; TYKERB®; Urelumab; VCR; Vectibix™; Velban®; Velcade®; Venetoclax; VePesid®; Vesanoid®; Viadur™; Vidaza®; Vinblastine; Vinblastine Sulfate; Vincasar Pfs®; Vincristine; Vinorelbine; Vinorelbine tartrate; VLB; VM-26; Vorinostat; Votrient; VP-16; Vumon®; Xeloda®; Zanosar®; Zevalin™; Zinecard®; Zoladex®; Zoledronic acid; Zolinza; or Zometa®, and/or any other agent not specifically listed here that target similar pathways.

When two or more substances or principles are to be used as part of a combined treatment regimen, they can be administered via the same route of administration or via different routes of administration, at essentially the same time or at different times (e.g. essentially simultaneously, consecutively, or according to an alternating regime). When the substances or principles are to be administered simultaneously via the same route of administration, they may be administered as different pharmaceutical formulations or compositions or part of a combined pharmaceutical formulation or composition, as will be clear to the skilled person.

Also, when two or more active substances or principles are to be used as part of a combined treatment regimen, each of the substances or principles may be administered in the same amount and according to the same regimen as used when the compound or principle is used on its own, and such combined use may or may not lead to a synergistic effect. However, when the combined use of the two or more active substances or principles leads to a synergistic effect, it may also be possible to reduce the amount of one, more than one, or all of the substances or principles to be administered, while still achieving the desired therapeutic action. This may, e,g., be useful for avoiding, limiting or reducing any unwanted side-effects that are associated with the use of one or more of the substances or principles when they are used in their usual amounts, while still obtaining the desired pharmaceutical or therapeutic effect.

The effectiveness of the treatment regimen used according to the invention may be determined and/or followed in any manner known per se for the disease or disorder involved, as will be clear to the clinician. The clinician will also be able, where appropriate and on a case-by-case basis, to change or modify a particular treatment regimen, so as to achieve the desired therapeutic effect, to avoid, limit or reduce unwanted side-effects, and/or to achieve an appropriate balance between achieving the desired therapeutic effect on the one hand and avoiding, limiting or reducing undesired side effects on the other hand.

Generally, the treatment regimen will be followed until the desired therapeutic effect is achieved and/or for as long as the desired therapeutic effect is to be maintained. Again, this can be determined by the clinician.

In various embodiments, pharmaceutical compositions comprising one or more OX40 receptor agonist proteins, either alone or in combination with prophylactic agents, therapeutic agents, and/or pharmaceutically acceptable carriers are provided herein. In various embodiments, nonlimiting examples of the uses of the pharmaceutical compositions disclosed herein include diagnosing, detecting, and/or monitoring a disorder, preventing, treating, managing, and/or ameliorating a disorder or one or more symptoms thereof, and/or in research. The formulation of pharmaceutical compositions, either alone or in combination with prophylactic agents, therapeutic agents, and/or pharmaceutically acceptable carriers, are known to one skilled in the art (US Patent Publication No. 20090311253 A1).

As used herein, the phrase “effective amount” means an amount of OX40L agonist protein that results in a detectable improvement (e.g., at least about 5%, 10%, 15%, 20%, 25%, 30%, 35%, 40%, 45%, 50%, 55%, 60%, 65%, 70%, 75%, or more from baseline) in one or more parameters associated with a dysfunction of OX40L or with a OX40L-associated disease or disorder.

Methods of administering a therapeutic agent provided herein include, but are not limited to, oral administration, parenteral administration (e.g., intradermal, intramuscular, intraperitoneal, intravenous and subcutaneous), epidural administration, intratumoral administration, mucosal administration (e.g., intranasal and oral routes) and pulmonary administration (e.g., aerosolized compounds administered with an inhaler or nebulizer). The formulation of pharmaceutical compositions for specific routes of administration, and the materials and techniques necessary for the various methods of administration are available and known to one skilled in the art (US Patent Publication No. 20090311253 A1).

In various embodiments, dosage regimens may be adjusted to provide for an optimum desired response (e.g., a therapeutic or prophylactic response). For example, a single bolus may be administered, several divided doses may be administered over time or the dose may be proportionally reduced or increased as indicated by the exigencies of the therapeutic situation. In some embodiments, parenteral compositions are formulated in dosage unit form for ease of administration and uniformity of dosage. The term “dosage unit form” refers to physically discrete units suited as unitary dosages for the mammalian subjects to be treated; each unit containing a predetermined quantity of active compound calculated to produce the desired therapeutic effect in association with the required pharmaceutical carrier.

An exemplary, non-limiting range for a therapeutically or prophylactically effective amount of a OX40 receptor agonist protein provided herein is about 0.1-100 mg/kg, (e.g., about 0,1-0.5, 0.1-1, 0.1-10, 0.1-20, 0,1-50, 0.1-75, 1-10, 1-15, 1-7.5, 1.25-15, 1.25-7.5, 2.5-7.5, 2.5-15, 5-15, 5-7.5,1-20, 1-50, 7-75, 1-100, 5-10, 5-15, 5-20, 5-25, 5-50, 5-75, 10-20, 10-50, 10-75, or 10-100 mg/kg, or any concentration in between). In some embodiments, the OX40 receptor agonist protein is present in a pharmaceutical composition at a therapeutically effective concentration, e.g., a concentration of about 0.1-100 mg/ml(e.g., about 0.1-0.5, 0.1-1, 0.1-10, 0.1-20, 0.1-50, 0.1-75, 1-10, 1-20, 1-50, 1-75, 1-100, 5-10, 5-15, 5-20, 5-25, 5-50, 5-75, 10-20, 10-50, 10-75, or 10-100 mg/ml, or any concentration in between). Note that dosage values may vary with the type and/or severity of the condition to be alleviated. It is to be further understood that for any particular subject, specific dosage regimens may be adjusted over time according to the individual need and/or the professional judgment of the person administering or supervising the administration of the compositions, and that dosage ranges set forth herein are exemplary only and are not intended to limit the scope or practice of the claimed composition.

### EXAMPLES

### Example 1

### Manufacture of a OX40 Receptor Agonist Protein

1.1 Polypeptide Structure

A) Amino acids Met1-Gly20
Ig-Kappa-signal peptide, assumed signal peptidase cleavage site after amino acid Gly 20.

B) Amino acids Arg21-Leu149
First soluble cytokine domain of the human OX40L ligand (OX40L, amino acid 55-133 of SEQ ID NO: 1).

C) Amino acids Gly150-Ser 157
First peptide linker element of SEQ ID NO: 2.

D) Amino acids Arg158-Leu286
Second soluble cytokine domain of the human OX40L ligand (OX40L, amino acid 55-133 of SEQ ID NO: 1).

E) Amino acids Gly287-Ser294.
Second peptide linker element of SEQ ID NO: 2.

F) Amino acids Arg295 Leu423
Third soluble cytokine domain of the human OX40L ligand (OX40L, amino acid 55-133 of SEQ ID NO: 1).

G) Amino acids Gly424-Cys444
Hinge-linker element of SEQ ID NO: 16.

H) Amino acids Pro445 Lys662
Antibody Fc fragment domain of SEQ ID NO: 13.

The above OX40 receptor agonist protein is shown in SEQ ID NO: 25.

The indicated linkers may be replaced by other preferred linkers, e.g. as shown in SEQ ID NOs: 3-12.

The indicated Hinge-linker element may be replaced by other preferred Hinge-linkers, e.g. as shown in SEQ ID NOs: 19-24.

It should be noted that the first and second peptide linkers do not need to be identical.

The signal peptide sequence (A) may be replaced by any other suitable, e.g., mammalian signal peptide sequence.

1.2 Gene Cassette Encoding the Polypeptide

The synthetic gene may be optimized in view of its codon usage for the expression in suitable host cells, e.g. insect cells or mammalian cells. A preferred nucleic acid sequence is shown in SEQ ID NO: 37.

### Example 2

### Expression and Purification

2.1 Cloning, Expression and Purification of Fusion Polypeptides

The aforementioned fusion proteins are expressed recombinantly in different eukaryotic host cells employing the methods described below:

Method for Small Scale Expression of OX40 Receptor Agonist Fusion Proteins:

For small scale analysis of aforementioned OX40 receptor agonist fusion proteins, Hek293 cells are grown in DMEM+GlutaMAX (GibCo) supplemented with 10% FBS, 100 units/ml Penicillin and 100 [mu]g/ml Streptomycin and are transiently transfected with a plasmid containing an expression cassette for a fusion polypeptide and an appropriate selection marker, e.g. a functional expression cassette comprising a blasticidine, puromycin or hygromycin resistence gene. In those cases, where a plurality of polypeptide chains is necessary to achieve the final product, the expression cassettes are either combined on one plasmid or positioned on different plasmids during the transfection. Cell culture supernatant containing recombinant fusion polypeptide are harvested three days post transfection and clarified by centrifugation at 300×g followed by filtration through a 0.22 μm sterile filter.

Method for Large Scale Expression and Purification of OX40 Receptor Agonist Fusion Proteins

For larger scale expression of OX40 receptor agonist fusion proteins, synthetic DNA cassettes encoding the aforementioned proteins are inserted into eukaryotic expression vectors comprising appropriate selection markers (e.g. a functional expression cassette comprising a blasticidin, puromycin or hygromycin resistance gene) and genetic elements suitable to enhance the number of transcriptionally active insertion sites within the host cells genome. The sequence verified expression vectors is introduced by electroporation into suspension adapted Chinese Hamster Ovary cells (CHO-S, Invitrogen). Appropriate selection pressure will be applied three days post-transfection to transfected cells. Surviving cells carrying the vector derived resistance gene(s) are recovered by subsequent cultivation under selection pressure. Upon stable growth of the selected cell pools in chemically defined medium (PowerCHO2-CD, Lonza) at 37° C. and 7% CO2 atmosphere in an orbital shaker incubator (100 rpm, 50 mm shaking throw), the individual supernatants are analyzed by ELISA-assays detecting the aforementioned proteins and the cell pools with the highest specific productivity are expanded in shake flasks prior to protein production (orbital shaker, 100 rpm, shaking throw 50 mm).

For lab-scale protein production, individual cell pools are cultured for 7-12 days in chemically defined medium (PowerCHO2-CD, Lonza) at 37° C. and 7% CO2 atmosphere in a Wave bioreactor 20/50 EHT (GE-Healthcare). The basal medium is PowerCHO2-CD supplemented with 4 mM Glutamax. Wave culture is started with a viable cell concentration of 0.3 to 0.4×10e6 cells/ml and the following settings (for a five- or ten liter bag): shaking frequency 18 rpm, shaking ankle 7°, gas current 0.2-0.3 L/min, 7% CO2, 36.5° C. During the Wave run, the cell culture is fed twice with PowerFeed A (Lonza), usually on day 2 (20% feed) and day 5 (30% feed). After the second feed, shaking frequency is increased to 22 rpm, as well as the shaking ankle to 8°.

The bioreactor is usually harvested in between day 7 to day 12 when the cell viability drops below 80%. First, the culture supernatant is clarified using a manual depth filtration system (Millipore Millistak Pod, MC0HC 0.054 m²). For Strep-tagged proteins, Avidin is added to a final concentration of 0.5 mg/L. Finally, the culture supernatant containing the OX40 receptor agonist fusion protein is sterile filtered using a bottle top filter (0.22 μm, PES, Corning) and stored at 2-8° C. until further processing.

For affinity purification Streptactin Sepharose is packed to a column (gel bed 2 ml), equilibrated with 15 ml buffer W (100 mM Tris-HCl, 150 mM NaCl, pH 8.0) or PBS pH 7.4 and the cell culture supernatant is applied to the column with a flow rate of approx. 4 ml/min. Subsequently, the column is washed with 15 ml buffer W and bound polypeptide is eluted stepwise by addition of 7×1 ml buffer E (100 mM Tris HCl, 150 mM NaCl, 2.5 mM Desthiobiotin, pH 8.0). Alternately, PBS pH 7.4 containing 2.5 mM Desthiobiotin can be used for this step.

Alternately to the Streptactin Sepharose based method, the affinity purification is performed employing a column with immobilized Protein-A as affinity ligand and an Akta chromatography system (GE-Healthcare). A solid phase material with high affinity for the FC-domain of the fusion protein is chosen: MABSelect Sure™ (GE Healthcare). Briefly, the clarified cell culture supernatant is loaded on a HiTrap MabSelectSure column (CV=5 ml) equilibrated in wash-buffer-1 (20 mM Pi, 95 mM NaCl, pH7.2) not exceeding a load of 10 mg fusion protein per ml column-bed. The column is washed with ten column-volumes (10CV) of aforementioned equilibration buffer followed by four column-volumes (4CV) of wash-buffer-2 (20 mM Pi, 95 mM NaCl, pH 8.0) to deplete host-cell protein and host-cell DNA. The column is then eluted with elution buffer (20 mM Pi, 95 mM NaCl, pH 3.5) and the eluate is collected in up to ten fractions with each fraction having a volume equal to column-bed volume (5 ml). Each fraction is neutralized with an equal volume of aforementioned wash-buffer-2. The linear velocity is set to 150 cm/h and kept constant during the aforementioned affinity chromatography method. The protein amount of the eluate fractions is quantitated and peak fractions are concentrated by ultrafiltration and further purified by size exclusion chromatography (SEC).

SEC is performed on Superdex 200 10/300 GL or HiLoad 26/60 columns using an Akta chromatography system (GE-Healthcare). The columns are equilibrated with phosphate buffered saline and the concentrated, affinity-purified polypeptide is loaded onto the SEC column with the sample volume not exceeding 2% (v/v) of the column-volume. In the case of Superdex 200 10/300 GL columns (GE Healthcare), a flow rate of 0.5 ml per minute is applied. In the case of HiLoad 26/60 Superdex200 columns, a flow rate of 2.5 ml per minute is applied. The elution profile of the polypeptide is monitored by absorbance at 280 nm.

For determination of the apparent molecular weight of purified fusion polypeptide under native conditions a Superdex 200 column is loaded with standard proteins of known molecular weight. Based on the elution volume of the standard proteins a calibration curve is plotted and the molecular weight of purified fusion polypeptide is determined. The FC-domain comprising OX40 receptor agonist fusion proteins elutes from the Superdex200 columns with an apparent molecular weight of approx. 140-180 kDa, which would confirm the homodimerisation of the mature OX40 receptor agonist fusion polypeptide by the Fc domain.

### Example 3

### Trivalent Control Protein

To compare the relative binding between hexavalent OX40 receptor agonist fusion proteins and the, homo-trimeric trivalent OX40 receptor agonist fusion proteins stabilized with bacteriophage RB69-FOLDON is expressed in CHO-S cells and purified as described in the former section. The sequence is shown in the table below:

| SEQ ID | |
|---|---|
| NO | Sequence |
| 38 | METDTLLVFVLLVWVPAGNGRYPRIQSIKVQFTEYKK |
| (Trivalent | EKGFILTSQKEDEIMKVQNNSVIINCDGFYLISLKGY |
| control | FSQEVNISLHYQKDEEPLFQLKKVRSVNSLMVASLTY |
| protein) | KDKVYLNVTTDNTSLDDFHVNGGELILIHQNPGEFCV |
| | LGSGSSGSSGSSGSGYIEDAPSDGKFYVRKDGAWVEL |
| | PTASGPSSSSSSAWSHPQFEK. |

### Example 4

### Determination of the In Vitro Stability of OX40 Receptor Agonist Proteins by Limited Protease Digestion

All OX4 receptor agonist proteins to be investigated will be expressed and purified as hexavalent Fc-Fusion protein as described in Example 1. The set will include OX40 receptor agonist proteins comprising the N297S mutation [according to the EU numbering system] in the CH2-domain and a hinge region that enables the formation of three disulfide bridges and additionally lack the upper hinge lysine [K223, according to the EU numbering system] which is mutated to glycine [K223G]. In a limited protease is digestion assay, the aforementioned OX40 receptor agonist proteins comprising the N297S mutation and the K223G mutation simultaneously in context of a three disulfide enabling hinge will be compared to OX40 receptor agonist proteins comprising the N297S mutation but have the K223 wildtype present either in the context of a two disulfide or three disulfide enabling hinge region.

In addition OX40 receptor agonist proteins with the second linker element (iv) reduced to 4 amino-acids and the shortened hinge element (vi) will be investigated (e.g. SEQ ID NO: 32 and 34). Both engineering strategies (N297S combined with K223G mutation in context of a three disulfide enabling hinge region) and shortage of linker elements (iv and vi) have a potential impact on the stability of the respective molecules.

The stability of different OX40 agonistic proteins of the present invention can be addressed by limited protease digestion in vitro. For this analysis, the aforementioned OX40 receptor agonist proteins are incubated with low concentrations of proteases (e.g. Trypsin, V8 protease) at different temperatures (e.g. 4° C., 25° C., 37° C.) for different amounts of time. Quantification of specific proteolytic fragments and their appearance over time can be subsequently measured by different methods, like SDS-PAGE, analytical SEC or analytical Mass-Spectrometry methods known in the art (e.g. Nano-RP-HPLC-ESI-MSMS). As the investigated proteins have most of their sequences in common, the faster appearance and enlarged quantities of specific proteolytic fragments from individual proteins over time can then be used to judge their relative stability and rank them to each other. With regard to protease based decoy kinetics of the aforementioned OX40 receptor agonist proteins investigated, the following order regarding their proteolytic stability is to be expected:

The OX40 receptor agonist proteins comprising the N2975 and the K223G and the three disulfide enabling hinge region simultaneously have a prolonged stability as compared to the OX40 receptor agonist proteins comprising the N2975 and wildtype K223 in the hinge region. The OX40 receptor agonist proteins comprising the SEQ ID NO: 21 as hinge linker have a prolonged stability as compared to OX40 receptor agonist proteins comprising the SEQ ID NO: 16 as hinge linker element.

### EXAMPLE 5

### Stability/Aggregation Test

The contents of monomers and aggregates are determined by analytical SEC as described in Example 2. For this particular purpose the analysis is performed in buffers containing physiological salt concentrations at physiological pH (e.g. 0.9% NaCl, pH 7.4; PBS pH 7.4). A typical aggregation analysis is done on a Superdex200 column (GE Healthcare). This column separates proteins in the range between 10 to 800 kDa.

For determination of the apparent molecular weight of purified fusion polypeptide under native conditions a Superdex 200 column is loaded with standard proteins of known molecular weight. Based on the elution volume of the standard proteins a calibration curve is plotted and the apparent molecular weight of purified fusion proteins of unknown molecular weight is calculated based on the elution volume.

SEC analysis of soluble, non-aggregated protein typically shows a distinct single protein peak at a defined elution volume (measured at OD at 280 nm or at OD 214 nm). This elution volume corresponds to the apparent native molecular weight of the particular protein. With regard to the definition of “monomer” in the case of FC-fusion proteins, the assembly of two polypeptide-chains is driven by the FC-part of the protein and the functional unit is a protein consisting of two chains. This unit that contains two FC-linked polypeptide chains is defined as “monomer” in the case of Fc-fusion proteins regardless of being a dimerized single-chain fusion polypeptide.

If protein aggregation occurs, the SEC analysis shows additional protein peaks with lower retention volumes. Protein oligomers potentially serve as aggregation seeds and a high content of oligomers potentially leads to aggregation of the protein. Oligomers of large molecular weight and aggregates elute in the void volume of the Superdex200 column and cannot be analyzed by SEC with respect to their native molecular weight.

Purified preparations of OX4 receptor agonist fusion proteins should preferably contain only defined monomeric protein and only a very low amount of oligomeric protein. The degree of aggregation/oligomerization of a particular OX40 receptor agonist fusion protein preparation is determined on basis of the SEC analysis by calculating the peak areas of the OD280 diagram for the defined monomer and the oligomer/aggregate fraction, respectively. Based on the total peak area the percentage of defined monomer protein is calculated as follows:

monomer content [%]=[Peak area monomer protein]/[Total peak area]×100)

### Example 6

### Determination of the Equilibrium Binding Constants for Tri- and Hexavalent OX40 Receptor Ligand Constructs by QCM Analysis

The equilibrium binding constants (K_{D}) of trivalent and hexavalent constructs of OX40 receptor ligand are calculated based on kinetic binding data (kₒₙ and k_{off}) that are determined with an automated biosensor system (Attana A100). The A100 allows to investigate molecular interactions in real-time based on the Quartz Crystal Microbalance (QCM) technique.

For this purpose the human OX40 receptor is immobilized to the surface of a carboxyl-activated QCM-chip. Subsequently the tri- or hexavalent OX40 receptor ligand, respectively, is used as an analyte at different concentrations (e.g. 0.5, 1, 2, 5, and 10 μg/ml) for analyzing the kinetic binding data for ligand-receptor binding (kₒₙ) and dissociation (k_{off}). The analysis is done in real time and the respective K_{D} can be calculated: K_{D}=k_{off}/kₒₙ.

The QCM analysis shows that the trivalent OX40 receptor ligand binds to the respective immobilized OX40 receptor with a K_{D} in the low nM-range with an expected K_{D} of 1-500 nM. However, hexavalent constructs of OX40 receptor ligand show a higher binding affinity in the pM-range towards the respective immobilized OX40 receptor with an expected K_{D} of 1 pM-500 nM. A common characteristic of the kinetic binding data (kₒₙ and k_{off}) is that the hexavalent constructs show faster kₒₙ in comparison to the trivalent constructs. In addition slower dissociation (k_{off}) is commonly observed for the hexavalent ligands if compared to the trivalent ligand.

### Example 7

### T Cell Proliferation Assay

To assess the T cell activation capability of the OX40 receptor agonist, T cells are purified from human buffy coat preparations by negative selection using magnetic beads. Cells are labeled with CFSE and incubated with or without varying amounts of the OX40 receptor agonist and combined with an anti-human CD3 antibody for 2-5 days at 37° C. Data on CFSE dilution as a means to measure cell division is acquired on a flow cytometer. IFNγ production is measured by an ELISA assay using cell culture supernatants and an anti-human IFNγ antibody for capture.

One expects to observe a clear augmentation of IFNγ secretion by both CD4+ and CD8+ T cells when the OX40 receptor agonist is present in the T cell cultures along with the anti-human CD3 antibody. As well as higher IFNγ production one expects to see more T cells to be driven into cell cycle by measuring CFSE dilution using flow cytometry. This would demonstrate a co-stimulatory effect of the OX40 receptor agonist in the context of T cell activation,

### Example 8

### OX40 Agonist Binding Assay

Primary, human T cells are isolated from fresh buffy coat preparations using negative selection and magnetic beads. Cells are seeded into 24-well plates at 2×10e6 cells per well. T cells are incubated with an anti-human CD3 antibody (clone HIT3a, 1 μg/ml), anti-human CD28 antibody (clone CD28.2, 5 μg/ml) and varying amounts of Protein A (OX40L, 10-1000 ng/ml) or simply left in medium as control. After 3 days at 37° C. cells are fluorescently labeled with anti-human OX40 and anti-human CD4 or anti-human is CD8 antibodies. OX40 fluorescence is assessed on a guava easyCyte flow cytometer within CD4+ and CD8+ T cell populations.

When comparing T cell populations incubated with anti-CD3 and anti-CD28 antibodies to control cells left in medium alone, one expects to observe a lower flourescent signal for OX40 indicating an activation-induced downregulation of the receptor. This effect can be stronger and dose-dependent, when cells are co-incubated with the OX40 agonist (Protein A), which indicates a supplementary effect caused by the OX40 agonist (Protein A). Such results would suggest a binding of the OX40 agonist (Protein A) to its receptor in vitro.

### Example 9

### Human In Vitro T Cell Proliferation Assay

Total T cells (human) purified by negative selection and magnetic beads (pan T cell isolation kit, Miltenyi Biotec) from the peripheral blood of healthy donors and stained with CFSE (CellTrace™ CFSE Cell Proliferation Kit, for flow cytometry, ThermoFisher) and seeded into 24-well plates at 2×10e6 cells per well. Cells were incubated at 37° C. for 5 days with media alone, soluble anti-CD3 antibody (clone OKT3 at 1 μg/ml) alone, anti-CD3 antibody plus anti-CD28 antibody (clone 28.2 at 1 μg/ml) or anti-CD3 antibody plus mature Protein A (SEQ ID NO: 27) at 10, 100 or 1000 ng/ml, respectively.

On day 5, cells were washed and stained with DAPI (to exclude dead cells) and specific antibodies. Expression of Forward Scatter (FSC or size) and CFSE dilution (a measurement of proliferation) was measured by flow cytometry with a Guava EasyCyte 12 Flow Cytometer (EMD Millipore). Data analysis was performed on a minimum of ten thousand recorded events per sample with FlowJo 10.1 software (FlowJo, LLC). The percentage of responding cells was determined by gating on Forward Scatter and CFSE using the media control to determine proper gate location. Cells that had either increased cell size or decreased CFSE levels were labeled as responding cells. The individual data from two biological replicates from one donor is shown in in the table (Quantification of T cell activation) below. These results are consistent with results from additional donors and clearly show that treatment of human T cells in vitro with PROTEIN A enhances T cell activation and proliferation as compared to antibody stimulation alone.

Quantification of T Cell Activation:

Human T cell activation following
treatment with PROTEIN A in vitro

% of cells responding

| Stimulation | Sample 1 | Sample 2 |
|---|---|---|
| Media | 3 | 3 |
| anti-CD3 | 56 | 62 |
| anti-CD3/28 | 87 | 85 |
| anti-CD3 + Protein A 10 ng/ml | 62 | 63 |
| anti-CD3 + Protein A 100 ng/ml | 72 | 67 |
| anti-CD3 + Protein A 1000 ng/ml | 69 | 72 |

### Example 10

### Receptor Binding Assay

For ELISA assays assessing functional binding of OX40L to its corresponding receptor, coating of microtiter plates was performed with 1 μg/ml OX40-Fc (Bio-Techne GmbH, Wiesbaden-Nordenstadt, Germany). After blocking with StartingBlock (Life Technologies GmbH, Darmstadt, Germany), wells were incubated with indicated concentrations of OX40L compound. OX40L bound to its corresponding receptor was detected via its Strep Tag II employing the anti-StrepTag-peroxidase StrepTactin-HRP (1:5000, IBA GmbH, Goettingen, Germany) and subsequent detection of the converted Peroxidase-substrate TMB one (Kem-En-Tec Diagnostics, Taastrup, Denmark) at a wavelength of 450 nm in an ELISA reader. FIG. 6 clearly depicts concentration dependent binding of Protein A to its receptor.

What is claimed is:

1. A OX40 receptor agonist protein comprising a single-chain fusion polypeptide comprising:
(i) a first soluble OX40L domain,
(ii) a first peptide linker,
(iii) a second soluble OX40L domain,
(iv) a second peptide linker, and
(v) a third soluble OX40L domain, and
(vi) a hinge-linker selected from the group comprising SEQ D NOs: 16 and 19-24, and
(vii) an antibody Fc fragment, wherein the antibody Fc fragment (vii) consists of the amino acid sequence as shown in SEQ ID NO: 13 or 14 or amino acids 1-217 of SEQ ID NO: 13 or 14.

2. The OX40 receptor agonist protein of claim 1, wherein the antibody Fc fragment (vii) is fused to the C-terminal end of the third OX40L domain (v) via a hinge-linker (vi).

3. The OX40 receptor agonist protein of claim 1, which is substantially non-aggregating.

4. The OX40 receptor agonist protein of claim 1, wherein the second and/or third soluble OX40L domain is an N-terminally shortened domain which optionally comprises amino acid sequence mutations.

5. The OX40 receptor agonist protein of claim 1, wherein at least one of the soluble OX40L domains, particularly at least one of the soluble OX40L domains (iii) and (v), is a soluble OX40L domain with an N-terminal sequence which starts with amino acid Q51 or R55, Y56, P57 or R58 of human OX40L according to SEQ ID NO: 1 and wherein Y56 may be replaced by a neutral amino acid, e.g. Ser or Gly.

6. The OX40 receptor agonist protein of claim 5, wherein at least one of the soluble OX40L domains, particularly at least one of the soluble OX40L domains (iii) and (v), is a soluble OX40L domain with an N-terminal sequence selected from
(a) P57-R58 and
(b) (Gly/Ser)56-R58.

7. The OX40 receptor agonist protein of claim 5, wherein the soluble OX40L domain ends with amino acid L183 of according to SEQ ID NO: 1 and/or optionally comprises a mutation at position Y69, L160, Q80, N90, C97, N114, E123, T144, Y145, K146, N152, N157, D162, H164, N166, G168, G178, F180 or C181 or at two or more of said positions.

8. The OX40 receptor agonist protein of claim 5, wherein at least the soluble OX40L domain (iii), is a C-terminal shortened OX40L domain ending with P177, G178, E179 or V182, and wherein the shortened domain optionally comprises mutations at C97 and/or C181.

9. The OX40 receptor agonist protein of claim 6, wherein the soluble OX40L domains (i), (iii) and (v) consist of amino acids 55-183 of human OX40L according to SEQ ID NO: 1.

10. The OX40 receptor agonist protein of claim 1, wherein the first and second peptide linkers (ii) and (iv) independently have a length of 3-8 amino acids, particularly a length of 3, 4, 5, 6, 7 or 8 amino acids, and preferably are glycine/serine linkers, optionally comprising an asparagine residue which may be glycosylated.

11. The OX40 receptor agonist protein of claim 10, wherein the first and the second peptide linkers (ii) and (iv) consist of the amino acid sequence according to SEQ ID NO: 2.

12. The OX40 receptor agonist protein of claim 1, which additionally comprises an N-terminal signal peptide domain, e.g. of SEQ ID NO: 17, which may comprise a protease cleavage site, and/or which additionally comprises a C-terminal element which may comprise and/or connect to a recognition/purification domain, e.g. a Strep-tag according to SEQ ID NO: 18.

13. The OX40 receptor agonist protein of claim 1, comprising the amino acid sequence of any one of SEQ ID NOs: 15 and 25-35.

14. The OX40 receptor agonist protein of claim 1, comprising two polypeptides each having the amino acid sequence as set forth in SEQ ID NOs: 27, 29, 30, 31, 32, 33, 34 or 35.

15. The OX40 receptor agonist protein of claim 14, wherein the two polypeptides are covalently linked through three interchain disulfide bonds formed at:
a) positions 415, 421, and 424 of SEQ ID NO: 27, 29, 30 or
b) positions 411, 417 and 420 of SEQ ID NO: 31, 35 or
c) positions 412, 418 and 421 of SEQ ID NO: 32, or
d) positions 410, 416 and 419 of SEQ ID NO: 33, or
e) positions 408, 414 and 417 of SEQ ID NO: 34.

16. The OX40 receptor agonist protein of claim 14, comprising one or more N-glycosylated asparagine residues selected from the list of N135 and N272 of SEQ ID NO: 27, 29, 30, 32, 35, and N134 and N269 of SEQ ID NO: 33, and N134 and N268 of SEQ ID NO: 34, and N135 of SEQ ID NO: 31.

17. The OX40 receptor agonist protein of claim 1, wherein the polypeptide(s) are further post-translationally modified.

18. The OX40 receptor agonist protein of claim 17, wherein the post-translational modification comprises modification of the N-terminal glutamine to pyroglutamate.

19. A nucleic acid molecule encoding an OX40 receptor agonist protein of claim 1.

20. An expression vector comprising the nucleic acid molecule of claim 19.

21. A cell or a non-human organism transformed or transfected with a nucleic acid molecule of claim 19, wherein the cell is a prokaryotic cell or a eukaryotic cell.

22. A pharmaceutical or diagnostic composition comprising as an active agent the OX40 receptor agonist protein of claim 1 and one or more pharmaceutically acceptable carriers, diluents, excipients and/or adjuvants.
